SEQUENCE LISTING (41 sequences)

SEQ ID NO: 1 - protein, 124 aa, Homo Sapiens
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSS

SEQ ID NO: 2 - protein, 124 aa, Homo Sapiens
QVQLVESGPGLVQPGGSLRISCAISGFSLTDYGVHWVRQAPGKGLEWLVVIWSDGSSTYNSALKSRMTISKDNSKSTVYLQMNSLRAEDTAMYYCARHGTYYGMTTTGDALDYWGQGTLVTVSS

SEQ ID NO: 3 - protein, 124 aa, Homo Sapiens
EVQLVESGGGLVQPGGSLRISCAISGFSLTDYGVHWVRQAPGKGLEWLVVIWSDGSSTYNSALKSRMTISKDNSKNTVYLQMNSLRAEDTAVYYCARHGTYYGMTTTGDALDYWGQGTLVTVSS

SEQ ID NO: 4 - protein, 124 aa, Homo Sapiens
EVQLVESGGGLVQPGGSLRLSCAASGFSLTDYGVHWVRQAPGKGLEWVSVIWSDGSSTYNSALKSRFTISRDNSKNTLYLQMNSLRAEDTAVYYCARHGTYYGMTTTGDALDYWGQGTLVTVSS

SEQ ID NO: 5 - protein, 124 aa, Homo Sapiens
EVQLVESGGGLVQPGGSLRLSCAISGFSLTDYGVHWVRQAPGKGLEWLVVIWSDGSSTYNSALKSRMTISKDNSKSTVYLQMNSLRAEDTAVYYCARHGTYYGMTTTGDALDYWGQGTLVTVSS

SEQ ID NO: 6 - protein, 124 aa, Homo Sapiens
QVQLVESGGGLVQPGGSLRISCAISGFSLTDYGVHWVRQAPGKGLEWLVVIWSDGSSTYNSALKSRMTISKDNSKSTVYLQMNSLRAEDTAMYYCARHGTYYGMTTTGDALDYWGQGTLVTVSS

SEQ ID NO: 7 - protein, 109 aa, Homo Sapiens
QIVLTQSPAIMSASLGERVTMTCTASSSVSSNYLHWYQQKPGSAPNLWIYSTSNLASGVPARFSGSGSGTSYSLTISSMEAEDAATYYCHQYLRSPPTFGGGTKLEIKR

SEQ ID NO: 8 - protein, 109 aa, Homo Sapiens
DIQLTQSPSSMSASLGDRVTMTCTASSSVSSNYLHWYQQKPGKAPNLWIYSTSNLASGVPSRFSGSGSGTDYTLTISSMQPEDFATYYCHQYLRSPPTFGQGTKLEIKR

SEQ ID NO: 9 - protein, 109 aa, Homo Sapiens
DIQLTQSPSSLSASVGDRVTMTCTASSSVSSNYLHWYQQKPGKAPKLWIYSTSNLASGVPSRFSGSGSGTDYTLTISSMQPEDFATYYCHQYLRSPPTFGQGTKLEIKR

SEQ ID NO: 10 - protein, 109 aa, Homo Sapiens
DIQMTQSPSSLSASVGDRVTITCTASSSVSSNYLHWYQQKPGKAPKLLIYSTSNLASGVPSRFSGSGSGTDFTLTISSLQPEDFATYYCHQYLRSPPTFGQGTKVEIKR

SEQ ID NO: 11 - protein, 109 aa, Homo Sapiens
DIQLTQSPSSLSASVGDRVTITCTASSSVSSNYLHWYQQKPGKAPKLWIYSTSNLASGVPSRFSGSGSGTDYTLTISSLQPEDFATYYCHQYLRSPPTFGQGTKVEIKR

SEQ ID NO: 12 - protein, 109 aa, Homo Sapiens
DIQLTQSPSSLSASVGDRVTMTCTASSSVSSNYLHWYQQKPGKAPKLWIYSTSNLASGVPSRFSGSGSGTDYTLTISSLQPEDFATYYCHQYLRSPPTFGQGTKVEIKR

SEQ ID NO: 13 - DNA, 429 bp, Homo Sapiens
atggctgtcctggggctgcttctctgcctggtgactttcccaagctgtgtcctgtcccaggtgcagctgaaggagtcaggacctggcctggtggcgccctcacagagcctgtccatcacatgcaccatctcagggttctcattaaccgactatggtgttcactgggttcgccagcctccaggaaagggtctggagtggctggtagtgatttggagtgatggaagctcaacctataattcagctctcaaatccagaatgaccatcaggaaggacaactccaagagccaagttttcttaataatgaacagtctccaaactgatgactcagccatgtactactgtgccagacatggaacttactacggtatgactacgacgggggatgctttggactactggggtcaaggaacctcagtcaccgtctcctca

SEQ ID NO: 14 - DNA, 390 bp, Homo Sapiens
atggattttcaggtgcagattttcagcttcctgctaatcagtgcctcagtcataatgtccagaggacaaattgttctcacccagtctccagcaatcatgtctgcatctctaggggaacgggtcaccatgacctgcactgccagttcaagtgtaagttccaattacttgcactggtaccagcagaagccaggatccgcccccaatctctggatttatagcacatccaacctggcttctggagtcccagctcgtttcagtggcagtgggtctgggacctcttactctctcacaatcagcagcatggaggctgaagatgctgccacttattactgccaccagtatcttcgttccccaccgacgttcggtggaggcaccaagctggaaatcaaa

SEQ ID NO: 15 - DNA, 429 bp, Homo Sapiens
atggctgtcctggggctgcttctctgcctggtgactttcccaagctgtgtcctgtcccaggtgcagctgaaggagtcaggacctggcctggtggcgccctcacagagcctgtccatcacatgcaccatctcagggttctcattaaccgactatggtgttcactgggttcgccagcctccaggaaagggtctggagtggctggtagtgatttggagtgatggaagctcaacctataattcagctctcaaatccagaatgaccatcaggaaggacaactccaagagccaagttttcttaataatgaacagtctccaaactgatgactcagccatgtactactgtgccagacatggaacttactacggtatgactacgacgggggatgctttggactactggggtcaaggaacctcagtcaccgtctcgagc

SEQ ID NO: 16 - protein, 143 aa, Homo Sapiens
MAVLGLLLCLVTFPSCVLSQVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSS

SEQ ID NO: 17 - DNA, 390 bp, Homo Sapiens
atggattttcaggtgcagattttcagcttcctgctaatcagtgcctcagtcataatgtccagaggacaaattgttctcacccagtctccagcaatcatgtctgcatctctaggggaacgggtcaccatgacctgcactgccagttcaagtgtaagttccaattacttgcactggtaccagcagaagccaggatccgcccccaatctctggatttatagcacatccaacctggcttctggagtcccagctcgtttcagtggcagtgggtctgggacctcttactctctcacaatcagcagcatggaggctgaagatgctgccacttattactgccaccagtatcttcgttccccaccgacgttcggtggaggcaccaagctcgagatcaaa

SEQ ID NO: 18 - protein, 130 aa, Homo Sapiens
MDFQVQIFSFLLISASVIMSRGQIVLTQSPAIMSASLGERVTMTCTASSSVSSNYLHWYQQKPGSAPNLWIYSTSNLASGVPARFSGSGSGTSYSLTISSMEAEDAATYYCHQYLRSPPTFGGGTKLEIK

SEQ ID NO: 19 - DNA, 459 bp, Homo Sapiens
tctagaccaccatggctgtcctggggctgcttctctgcctggtgactttcccaagctgtgtcctgtcccaggtgcagctgaaggagtcaggacctggcctggtggcgccctcacagagcctgtccatcacatgcaccatctcagggttctcattaaccgactatggtgttcactgggttcgccagcctccaggaaagggtctggagtggctggtagtgatttggagtgatggaagctcaacctataattcagctctcaaatccagaatgaccatcaggaaggacaactccaagagccaagttttcttaataatgaacagtctccaaactgatgactcagccatgtactactgtgccagacatggaacttactacggaatgactacgacgggggatgctttggactactggggtcaaggaacctcagtcaccgtctcctcaggtaagaatggcctctaga

SEQ ID NO: 20 - protein, 143 aa, Homo Sapiens; identical to SEQ ID NO: 16

SEQ ID NO: 21 - DNA, 425 bp, Homo Sapiens
acgcgtccaccatggattttcaggtgcagattttcagcttcctgctaatcagtgcctcagtcataatgtccagaggacaaattgttctcacccagtctccagcaatcatgtctgcatctctaggggaacgggtcaccatgacctgcactgccagttcaagtgtcagttccaattacttgcactggtaccagcagaagccaggatccgcccccaatctctggatttatagcacatccaacctggcttctggagtcccagctcgtttcagtggcagtgggtctgggacctcttactctctcacaatcagcagcatggaggctgaagatgctgccacttattactgccaccagtatcttcgttccccaccgacgttcggtggaggcaccaagctggaaatcaaacgtaagtagaatccaaagtctaga

SEQ ID NO: 22 - protein, 130 aa, Homo Sapiens; identical to SEQ ID NO: 18

SEQ ID NO: 23 - DNA, 1353 bp, Homo Sapiens
caggtgcagctgaaggagtcaggacctggcctggtggcgccctcacagagcctgtccatcacatgcaccatctcagggttctcattaaccgactatggtgttcactgggttcgccagcctccaggaaagggtctggagtggctggtagtgatttggagtgatggaagctcaacctataattcagctctcaaatccagaatgaccatcaggaaggacaactccaagagccaagttttcttaataatgaacagtctccaaactgatgactcagccatgtactactgtgccagacatggaacttactacggaatgactacgacgggggatgctttggactactggggtcaaggaacctcagtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatcatgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtctccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggtaaa

SEQ ID NO: 24 - DNA, 645 bp, Homo Sapiens
caaattgttctcacccagtctccagcaatcatgtctgcatctctaggggaacgggtcaccatgacctgcactgccagttcaagtgtaagttccaattacttgcactggtaccagcagaagccaggatccgcccccaatctctggatttatagcacatccaacctggcttctggagtcccagctcgtttcagtggcagtgggtctgggacctcttactctctcacaatcagcagcatggaggctgaagatgctgccacttattactgccaccagtatcttcgttccccaccgacgttcggtggaggcaccaagctggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 25 - protein, 451 aa, Homo Sapiens
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 215 aa, Homo Sapiens
QIVLTQSPAIMSASLGERVTMTCTASSSVSSNYLHWYQQKPGSAPNLWIYSTSNLASGVPARFSGSGSGTSYSLTISSMEAEDAATYYCHQYLRSPPTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - DNA, 696 bp, Homo Sapiens
caggtgcagctgaaggagtcaggacctggcctggtggcgccctcacagagcctgtccatcacatgcaccatctcagggttctcattaaccgactatggtgttcactgggttcgccagcctccaggaaagggtctggagtggctggtagtgatttggagtgatggaagctcaacctataattcagctctcaaatccagaatgaccatcaggaaggacaactccaagagccaagttttcttaataatgaacagtctccaaactgatgactcagccatgtactactgtgccagacatggaacttactacggaatgactacgacgggggatgctttggactactggggtcaaggaacctcagtcaccgtctcctcagcttccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccatca

SEQ ID NO: 28 - protein, 232 aa, Homo Sapiens
QVQLKESGPGLVAPSQSLSITCTISGFSLTDYGVHWVRQPPGKGLEWLVVIWSDGSSTYNSALKSRMTIRKDNSKSQVFLIMNSLQTDDSAMYYCARHGTYYGMTTTGDALDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPS

SEQ ID NO: 29 - DNA, 6 bp, Homo Sapiens
ctcgag

SEQ ID NO: 30 - DNA, 6 bp, Homo Sapiens
tctaga

SEQ ID NO: 31 - DNA, 6 bp, Homo Sapiens
acgcgt

SEQ ID NO: 32 - DNA, 35 bp, Homo Sapiens
ttttctagaccaccatggctgtcctggggctgctt

SEQ ID NO: 33 - DNA, 47 bp, Homo Sapiens
ttttctagaggttgtgaggactcacctgaggagacggtgactgaggt

SEQ ID NO: 34 - DNA, 31 bp, Homo Sapiens
tggaacttactacggaatgactacgacgggg

SEQ ID NO: 35 - DNA, 31 bp, Homo Sapiens
ccccgtcgtagtcattccgtagtaagttcca

SEQ ID NO: 36 - DNA, 43 bp, Homo Sapiens
ttttctagaggccattcttacctgaggagacggtgactgaggt

SEQ ID NO: 37 - DNA, 35 bp, Homo Sapiens
tttacgcgtccaccatggattttcaggtgcagatt

SEQ ID NO: 38 - DNA, 49 bp, Homo Sapiens
ttttctagattaggaaagtgcacttacgtttgatttccagcttggtgcc

SEQ ID NO: 39 - DNA, 31 bp, Homo Sapiens
tgccagttcaagtgtcagttccaattacttg

SEQ ID NO: 40 - DNA, 31 bp, Homo Sapiens
caagtaattggaactgacacttgaactggca

SEQ ID NO: 41 - DNA, 48 bp, Homo Sapiens
ttttctagactttggattctacttacgtttgatttccagcttggtgcc

(19) United States
(12) Patent Application Publication
(10) Pub. No.: US 2005/0002930 A1
(43) Pub. Date: Jan. 6, 2005

(54) Methods of production and use of anti-integrin antibodies for the control of tissue granulation

(76) Inventors: Dale E. Johnson, Emeryville, CA (US); Ursula Jeffry, Redwood City, CA (US)

Correspondence Address: HOWREY SIMON ARNOLD & WHITE, LLP;C/O M.P. DROSOS, DIRECTOR OF IP ADMINISTRATION, 2941 FAIRVIEW PK, BOX 7, FALLS CHURCH, VA, 22042, US

(21) Appl. No.: 10/818,068

(22) Filed: Apr. 2, 2004

Related U.S. Application Data
(60) Provisional application No. 60/460,642, filed on Apr. 3, 2003.

(51) Int. Cl. A61K 39/395

(52) U.S. Cl. 424/144.1

(57) ABSTRACT

The present invention provides methods that enable the user to identify inhibitors of tissue granulation in and around a wound site, thereby limiting excessive scar formation as the wounded tissue heals. The some granulation inhibitors identified using the methods of the invention inhibit granulation in and around a wound site up to five fold, with a corresponding decrease in the formation of scar tissue when tested on retinal injuries. Granulation inhibitors that can be identified using the methods of the present invention include antibodies, peptides, nucleic acids (aptamers), and non-peptide small molecules.

### RELATED APPLICATIONS

This application claims priority from U.S. Provisional Application No. 60/460,642 filed Apr. 3, 2003, which is hereby incorporated by reference herein in its entirety.

This application also is related to U.S. Pat. No. 10/724,274 filed Nov.26, 2003, which is hereby incorporated by reference herein in its entirety.

### FIELD OF THE INVENTION

The present invention relates to the field of biochemistry, and physiology, particularly to methods of enhancing wound healing. The methods provided enable the user to identify inhibitors useful as therapeutic agents to treat tissue granulation in and around a wound site, thereby limiting excessive scar formation as the wounded tissue heals. The granulation inhibitors identified using the methods of the invention inhibit granulation in and around a wound site up to five fold, with a corresponding decrease in the formation of scar tissue when tested on retinal injuries. In addition, these inhibitors inhibit macrophage behavior associated with lesions in RPE cells. Granulation inhibitors that can be identified using the methods of the present invention include antibodies, peptides, nucleic acids (aptamers), and non-peptide small molecules.

### BACKGROUND OF THE INVENTION

Wound repair and tissue generation in normal and impaired wound healing conditions is a major focus in medicine. A particular problem in wound healing is the scarring and tissue detachment from underlying membranes caused by fluid accumulation resulting from excessive granulation in and around the wound site. These problems are particularly acute in wounds to the eye and other tissues, such as joint cartilage. For example, in wound healing it has been shown that there is a major reorganization of collagen types I and III. The accumulation of such molecules in connective tissue is associated with diseases such as rheumatoid arthritis and atherosclerosis.

Thus, there is a need in the art for methods and compositions useful in controlling granulation in treated wounds, as well as repair in injured or grafted mammalian, particularly human, tissue.

### SUMMARY OF THE INVENTION

The present invention provides methods for controlling granulation in the region of injured tissue. In this manner, methods of the invention aid in minimizing tissue damage collateral to an initial injury. Accordingly, the present invention provides methods of reducing deleterious granulation that involve applying a granulation inhibitor to an injured or diseased tissue. The diseased or injured tissue may be part of an eye, joint or associated with a bursae. Some methods are useful for treating injured or diseased tissue produced by a condition such as keloid formation, burns or scleroderma. Other methods provide treatment for injured or diseased tissue associated with a disease causing tissue inflammation. Exemplary diseases of this type include f rheumatoid Arthritis, Wegener's Granulomatosis, Churg-Strauss-allergic granulomatosis, eosinophilic granulomata, midline granuloma, desmoid, sarcoidosis, macular degeneration, proliferative vitreoretinopathy, proliferative diabetic retinopathy, uterine fibroids, arteritis temporalis and Takayasu's arteritis. Diseases involving fibrosis resulting from inflammation also respond to the treatments described herein, for example, Crohn's disease, idiopathic pulmonary fibrosis, and allergic pulmonary fibrosis. Granulation inhibitors useful as medicaments in treating diseases such as those described above include antibodies, small organic molecules, and nucleic acid, protein and peptide antagonists.

Another embodiment of the present invention is methods for reducing granulation in an injured or diseased tissue that involves applying an α5β1 integrin binding agent to the tissue. Tissues responsive to these methods include eye, skin, bone, cartilage, vascular, ligaments and tendons. The binding agent may be applied to the injured or diseased tissue by a number of techniques, including direct application, intravitreal injection, systemic injection, nebulized inhalation, eye drop, and oral ingestion.

Tissue injuries that may be treated using methods of the invention include physical injuries, such as cuts, burns, bruises and punctures, chemical trauma, exposure to radiation sources and the like. Infectious diseases resulting in tissue damage may also be treated with the invention. The invention is however particularly suited for use in treating non-infectious diseases, most preferably in the treatment of injuries resulting in a sterile environment, such as during surgery, or occurring in a manner not likely to be accompanied by advantageous infections. Diseases treatable by the methods of the present invention include, but are not limited to, diabetic retinopathy, rheumatoid arthritis, osteoarthritis, macular degeneration by tissue granulation, temporal arteritis, polymyalgia rheumatica, giant cell arteritis, Takayasu's arteritis, Kawasaki's disease, Wegener's granulomatosis, Churg-Strauss alleric granulomatosis and angiitis, idiopathic pulmonary fibrosis, systemic sclerosis/scleroderma, Sjogren's syndrome/disease, sicca syndrome, allergic pulmonary fibrosis, sarcoidosis, uterine fibroids, hemangioma, lymphangioma, keloid scars formation, Goodpasteur disease, Crohns disease, Pagets syndrome, pterygiae, eosinophilic granulomata, autoimmune diseases that cause cellular granulation, and many injuries and diseases that induce neoangiogenesis in the affected tissue.

Some aspects of the invention use α5β1 integrin binding agents that are nucleic acids (aptamers) glycoproteins, small organic molecules, mutiens and the like. Most preferably the binding agent is an anti-α5B 1 integrin antibody, ideally an antibody having a variable heavy chain region having an amino acid sequence homologous to an amino acid sequence selected from the group consisting of SEQ ID NOS.: 1-6, and a variable light chain region having an amino acid sequence homologous to an amino acid sequence selected from the group consisting of SEQ ID NOS.: 7-12.

The present invention also includes methods for identifying inhibitors of cellular granulation. Some of these methods involve incubating a first wound tissue in the presence of an inhibitor candidate and a second wound tissue in the absence of the inhibitor candidate, and determining the level of cellular granulation present in the second wound tissue relative to the first wound tissue.

Other methods for identifying inhibitors of cellular granulation include additional screening. The additional screening involves incubating α5β1 integrin with a binding candidate; adding fibronectin to the α5β1 integrin, binding candidate incubation and determining if the α5β1 integrin binds the fibronectin. Failure to observe binding of α5β1 integrin to fibronectin indicates the binding candidate is an inhibitor candidate. Once inhibitor candidates are identified, they are tested for an ability to inhibit cellular granulation. This involves incubating a first wound tissue in the presence of the inhibitor candidate and a second wound tissue in the absence of the inhibitor candidate, and determining the level of cellular granulation present in the second wound tissue relative to the first wound tissue.

In some aspects of both methods for identifying inhibitors of cellular granulation the first and second wound tissues are eye tissue. In other aspects the determining step comprises examining stained tissue sections. Additional aspects include those where the binding or inhibitor candidate is a protein, preferably an anti-α5β1 integrin antibody, most preferably an antibody that comprises a variable heavy chain region having an amino acid sequence homologous to an amino acid sequence selected from the group consisting of SEQ ID NOS.: 1-6, and a variable light chain region having an amino acid sequence homologous to an amino acid sequence selected from the group consisting of SEQ ID NOS.: 7-12.

An embodiment relating to eye injuries and diseases provides a method of controlling RPE cell behavior that includes contacting a wound site in an affected eye with one of the α5β1 integrin binding agents described above. This results in RPE cells of the affected eye being inhibited from displaying macrophage behavior. Instead, the RPE cells appear to take on a more fibroblast-type morphology. The types of macrophage behavior inhibited include phagocytic activity, and secretion of cytokines, chemokines and mediators of inflammatory responses. Preferably the binding agent is an anti-(α5 1β1 integrin antibody, more preferably an antibody that binds competitively for a5b 1 integrin with antibody having a variable heavy chain region having an amino acid sequence homologous to an amino acid sequence selected from the group consisting of SEQ ID NOS.: 1-6, and a variable light chain region having an amino acid sequence homologous to an amino acid sequence selected from the group consisting of SEQ ID NOS.: 7-12, most preferably an antibody having a variable heavy chain region having an amino acid sequence homologous to an amino acid sequence selected from the group consisting of SEQ ID NOS.: 1-6, and a variable light chain region having an amino acid sequence homologous to an amino acid sequence selected from the group consisting of SEQ ID NOS.: 7-12. In some aspects of this embodiment the binding agent can be applied to the injured or diseased tissue by a number of methods including direct application to the injured or diseased tissues, intravitreal injection, systemic injection, nebulized inhalation, eye drop, and oral ingestion. Preferably wound site(s) of the injured or diseased eye are not created by an infection.

Eye tissue may also be used in methods to evaluate physiological effects modulated by a granulation inhibitor. These methods involve creating lesions in an eye tissue sufficient to produce granulation; applying one or more doses of a granulation inhibitor to the eye tissue, and monitoring granulation in or around the lesions of the dosed eye tissue. In some aspects of this embodiment the eye tissue is a part of the eye of a living primate. The eye tissue used in this embodiment can be retinal, macular or corneal. In some aspects of the invention creating lesions in the eye tissue is performed with laser light. In some aspects, the laser light is from about 300 to about 700 mwatts, and the exposure time is no more than 0.1 seconds. Preferably the lesions created are from about 50 to about 100μm in diameter.Application of the granulation inhibitor can be by any of the methods previously described, e.g., direct application, intravitreal injection, systemic injection, nebulized inhalation, eye drop, or oral ingestion. Although the granulation inhibitor can be any molecule previously mentioned, it is preferably an anti-α5β1 integrin antibody, more preferably an antibody having a variable heavy chain region having an amino acid sequence homologous to an amino acid sequence selected from the group consisting of SEQ ID NOS.: 1-6, and a variable light chain region having an amino acid sequence homologous to an amino acid sequence selected from the group consisting of SEQ ID NOS.: 7-12.

### BRIEF DESCRIPTION OF THE DRAWINGS

FIG. 1 depicts the amino acid sequences (SEQ ID NOS: 1-12) for the variable regions of the heavy (V_{H}) and light chains (V_{L}) of a murine anti-α5β1 integrin antibody (IIA1) and five humanized antibodies derived from the murine original (1.0-5.0)

FIG. 2 depicts an alignment of amino acid sequences (SEQ ID NOS: 1-12) that highlights sequence substitutions in the five humanized antibodies relative to the murine original (IIA1).

FIG. 3 depicts: (A) IIA1 V_{H} nucleic acid sequence (SEQ ID NO: 13) and amino acid sequence (SEQ ID NO: 1); (B) IIA1 V_{L} nucleic acid sequence (SEQ ID NO: 14) and amino acid sequence (SEQ ID NO: 7).

FIG. 4 depicts: (A) Antibody 200-4 V_{H} nucleic acid sequence (SEQ ID NO: 15) and amino acid sequence (SEQ ID NO: 16); (B) Antibody 200-4 V_{L} nucleic acid sequence (SEQ ID NO: 17) and amino acid sequence (SEQ ID NO: 18).

FIG. 5 depicts: (A) M200 V_{H} nucleic acid sequence (SEQ ID NO: 19) and amino acid sequence (SEQ ID NO: 20); (B) M200 V_{L} nucleic acid sequence (SEQ ID NO: 21) and amino acid sequence (SEQ ID NO: 22).

FIG. 6 depicts the p200-M-H plasmid construct for expression of M200 heavy chain.

FIG. 7 depicts the p200-M-L plasmid construct for expression of M200 light chain.

FIG. 8 depicts the single plasmid p200-M for expression of M200 heavy and light chains.

FIG. 9 depicts the complete M200 heavy chain and light chain DNA sequences (SEQ ID NOS: 23-24).

FIG. 10 depicts the complete M200 heavy chain and light chain amino acid sequences (SEQ ID NOS: 25-26).

FIG. 11 depicts the complete F200 heavy chain DNA and amino acid sequences (SEQ ID NOS: 27-28).

FIG. 12 depicts the decrease in scar tissue formation in the presence versus the absence of the granulation inhibitor F200 (also referred to “EOS200-F”).

FIG. 13A-13C depict the serum levels over the indicated period of the granulation inhibitor M200 (also referred to as “EOS200-4”) after a single intravenous injection of 5, 15 or 50 mg/kg body weight, respectively.

FIG. 13D-13F depict the serum levels over the indicated period of the granulation inhibitor M200 during a schedule of weekly intravenous injection of 5, 15 or 50 mg/kg body weight, respectively.

FIG. 14 is a summary of the results of a FACS study of whole blood taken from the individuals described in FIGS. 13A-13C showing the percent occupancy of blood monocytes α5β1 integrin binding sites by the granulation inhibitor M200. (Vehicle=0 mg/kg).

FIG. 15 is a summary of the results of a FACS study of whole blood taken from the individuals described in FIGS. 3A-3C showing the percent availability of blood monocytes α5β1 integrin binding sites. The data correlate with the data presented in FIG. 4, and indicate an inability of an anti-α5β1 antibody to bind monocytes α5β1 integrin binding sites taken from individuals dosed with the granulation inhibitor M200. (Vehicle=0 mg/kg).

### DETAILED DESCRIPTION OF THE INVENTION

I. Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The term “integrin” refers to extracellular receptors that are expressed in a wide variety of cells and bind to specific ligands in the extracellular matrix. The specific ligands bound by integrins may contain an arginine-glycine-aspartic acid tripeptide (Arg-Gly-Asp; RGD) or a leucine-aspartic acidvaline tripeptide, and include, for example, fibronectin, vitronectin, osteopontin, tenascin, and von Willebrand's factor. The integrins area superfamily of heterodimers composed of an α subunit and a β subunit. Numerous a subunits, designated, for example, αV, α5 and the like, and numerous β subunits, designated, for example, β1, β2, β3, β5 and the like, have been identified, and various combinations of these subunits are represented in the integrin superfamily, including α5β1, αVβ3 and αVβ5. The superfamily of integrins can be subdivided into families, for example, as αV-containing integrins, including αVβ3 and αVβ5, or the Pi-containing integrins, including α5β1 and αVβ1. Integrins are expressed in a wide range of organisms, including C. elegans, Drosophila sp., amphibians, reptiles, birds, and mammals including humans.

As disclosed herein, proteins, particularly antibodies, muteins, nucleic acid aptamers, and peptide and nonpeptide small organic molecules that bind of α5β1 integrin may serve as “binding agents” and “granulation inhibitors” of the present invention. The term “binding agent” is used herein to mean an agent that can interfere with the specific interaction of a receptor and its ligand. An anti-α5β1 integrin antibody, which can interfere with the binding of α5β1 with fibronectin, or other α5β1 integrin ligand, thereby reducing or inhibiting the association, is an example of an α5β1 binding agent. An α5β1 binding agent can act as a competitive inhibitor or a noncompetitive inhibitor of α5β1 integrin binding to its ligand.

Granulation inhibitors include those binding agents that reduce tissue granulation when applied to a wound site, as described herein.

“Binding candidate” refers to molecular species that may specifically bind to α5β1 integrin, as defined herein. (I.e., a molecular species that may be a binding agent.)

“Inhibitor candidate” refers to molecular species that specifically bind to α5β1 integrin, and may inhibit cellular granulation when applied to wound tissue.

The phrase “specifically (or selectively) binds” or when referring to an antibody interaction, “specifically (or selectively) immunoreactive with,” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antibodies bind to a particular protein sequence, thereby identifying its presence.

Specific binding to an antibody under such conditions requires an antibody that is selected for its specificity for a particular protein. For example, antibodies raised against a particular protein, polymorphic variants, alleles, orthologs, and conservatively modified variants, or splice variants, or portions thereof, can be selected to obtain only those polyclonal antibodies that are specifically immunoreactive with α5β1 integrin and not with other proteins. This selection may be achieved by subtracting out antibodies that cross-react with other molecules. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods for determining whether two molecules specifically interact are disclosed herein, and methods of determining binding affinity and specificity are well known in the art (see, for example, Harlow and Lane, Antibodies: A laboratory manual (Cold Spring Harbor Laboratory Press, 1988); Friefelder, “Physical Biochemistry: Applications to biochemistry and molecular biology” (W.H. Freeman and Co. 1976)).

Generally binding agents and granulation inhibitors “interfere,” with α5β1 integrin binding to its natural ligands. “Interfere,” when used in reference to the action of a binding agent on the integrin-binding ability of another integrin ligand, means that the affinity of the interaction between integrin and its ligand is decreased below the level of binding that occurs in the absence of the binding agent. The skilled artisan will recognize that the association of a receptor and its ligand is a dynamic relationship that occurs among a population of such molecules such that, at any particular time, a certain proportion of receptors and ligands will be in association. An agent that interferes with the specific interaction of a receptor and its ligand, therefore, reduces the relative number of such interactions occurring at a given time and, in some cases, can completely inhibit all such associations. It can be difficult to distinguish whether an α5β1 integrin binding agent completely inhibits the association of a receptor with its ligand or reduces the association below the limit of detection of a particular assay. Thus, the term “interfere” is used broadly herein to encompass reducing or inhibiting the specific binding of a receptor and its ligand.

Furthermore, an α5β1 integrin binding agent can interfere with the specific binding of a receptor and its ligand by various mechanism, including, for example, by binding to the ligand binding site, thereby interfering with ligand binding; by binding to a site other than the ligand binding site of the receptor, but sterically interfering with ligand binding to the receptor; by binding the receptor and causing a conformational or other change in the receptor, which interferes with binding of the ligand; or by other mechanisms. Similarly, the agent can bind to or otherwise interact with the ligand to interfere with its specifically interacting with the receptor. For purposes of the methods disclosed herein, an understanding of the mechanism by which the interference occurs is not required and no mechanism of action is proposed. An α5β1 binding agent, such as an anti-α5β1 antibody, or antigen binding fragment thereof, is characterized by having specific binding activity (Kₐ)for an α5β1 integrin of at least about 10⁵ mol⁻¹, 10⁶ mol⁻¹ or greater, preferably 10⁷ mol⁻¹ l or greater, more preferably 10⁸ mol⁻¹ or greater, and most preferably 10⁹ mol⁻¹ or greater. The binding affinity of an antibody can be readily determined by one of ordinary skill in the art, for example, by Scatchard analysis (Scatchard, Ann. NY Acad. Sci. 51: 660-72, 1949).

The term “antibody” as used herein encompasses naturally occurring antibodies as well as non-naturally occurring antibodies, including, for example, single chain antibodies, chimeric, bifunctional and humanized antibodies, as well as antigen-binding fragments thereof, (e.g., Fab′, F(ab′)₂, Fab, Fv and rIgG). See also, Pierce Catalog and Handbook, 1994-1995 (Pierce Chemical Co., Rockford, Ill.). See also, e.g., Kuby, J., Immunology, 3^{rd} Ed., W.H. Freeman & Co., New York (1998). Such non-naturally occurring antibodies can be constructed using solid phase peptide synthesis, can be produced recombinantly or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); Borrabeck, Antibody Engineering, 2d ed. (Oxford University Press 1995); each of which is incorporated herein by reference).

The term “antibody” includes both polyclonal and monoclonal antibodies. The term also includes genetically engineered forms such as chimeric antibodies (e.g., humanized murine antibodies) and heteroconjugate antibodies (e.g., bispecific antibodies). The term also refers to recombinant single chain Fv fragments (scFv). The term antibody also includes bivalent or bispecific molecules, diabodies, triabodies, and tetrabodies. Bivalent and bispecific molecules are described in, e.g., Kostelny et al. (1992) J Immunol 148:1547, Pack and Pluckthun (1992) Biochemistry 31:1579, Hollinger et al., 1993, supra, Gruber et al. (1994) J Immunol :5368, Zhu et al. (1997) Protein Sci 6:781, Hu et al. (1996) Cancer Res. 56:3055, Adams et al. (1993) Cancer Res. 53:4026, and McCartney, et al. (1995) Protein Eng. 8:301.

Typically, an antibody has a heavy and light chain. Each heavy and light chain contains a constant region and a variable region, (the regions are also known as “domains”). Light and heavy chain variable regions contain four “framework” regions interrupted by three hypervariable regions, also called “complementarity-determining regions” or “CDRs”. The extent of the framework regions and CDRs have been defined. The sequences of the framework regions of different light or heavy chains are relatively conserved within a species. The framework region of an antibody, that is the combined framework regions of the constituent light and heavy chains, serves to position and align the CDRs in three dimensional space.

The CDRs are primarily responsible for binding to an epitope of an antigen. The CDRs of each chain are typically referred to as CDR1, CDR2, and CDR3, numbered sequentially starting from the N-terminus, and are also typically identified by the chain in which the particular CDR is located. Thus, a V_{H} CDR3 is located in the variable domain of the heavy chain of the antibody in which it is found, whereas a V_{L} CDR1 is the CDR1 from the variable domain of the light chain of the antibody in which it is found.

References to “V_{H}” refer to the variable region of an immunoglobulin heavy chain of an antibody, including the heavy chain of an Fv, scFv, or Fab. References to “V_{L”} refer to the variable region of an immunoglobulin light chain, including the light chain of an Fv, scfv, dsFv or Fab.

The phrase “single chain Fv” or “scFv” refers to an antibody in which the variable domains of the heavy chain and of the light chain of a traditional two chain antibody have been joined to form one chain. Typically, a linker peptide is inserted between the two chains to allow for proper folding and creation of an active binding site.

A “chimeric antibody” is an immunoglobulin molecule in which (a) the constant region, or a portion thereof, is altered, replaced or exchanged so that the antigen binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity.

A “humanized antibody” is an immunoglobulin molecule that contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin (Jones et al., Nature 321:522-525 (1986); Riechmann et al., Nature 332:323-329 (1988); and Presta, Curr. Op. Struct. Biol. 2:593-596 (1992)). Humanization can be essentially performed following the method of Winter and co-workers (Jones et al., Nature 321:522-525 (1986); Riechmann et al., Nature 332:323-327 (1988); Verhoeyen et al., Science 239:1534-1536 (1988)), by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species.

“Epitope” or “antigenic determinant” refers to a site on an antigen to which an antibody binds. Epitopes can be formed both from contiguous amino acids or noncontiguous amino acids juxtaposed by tertiary folding of a protein. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols in Methods in Molecular Biology, Vol. 66, Glenn E. Morris, Ed (1996). A preferred method for epitope mapping is surface plasmon resonance, which has been used to identify preferred granulation inhibitors recognizing the same epitope region as the IIAI antibody disclosed herein.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers, those containing modified residues, and non-naturally occurring amino acid polymer.

The term “amino acid” refers to naturally occurring and synthetic amino acids, as well as amino acid analogs and amino acid mimetics that function similarly to the naturally occurring amino acids. Naturally occurring amino acids are those encoded by the genetic code, as well as those amino acids that are later modified, e.g., hydroxyproline, γ-carboxyglutamate, and O-phosphoserine. Amino acid analogs refers to compounds that have the same basic chemical structure as a naturally occurring amino acid, e.g., an α carbon that is bound to a hydrogen, a carboxyl group, an amino group, and an R group, e.g., homoserine, norleucine, methionine sulfoxide, methionine methyl sulfonium. Such analogs may have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions similarly to a naturally occurring amino acid.

Amino acids may be referred to herein by their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical or associated, e.g., naturally contiguous, sequences. Because of the degeneracy of the genetic code, a large number of functionally identical nucleic acids encode most proteins. For instance, the codons GCA, GCC, GCG and GCU all encode the amino acid alanine. Thus, at every position where an alanine is specified by a codon, the codon can be altered to another of the corresponding codons described without altering the encoded polypeptide. Such nucleic acid variations are “silent variations,” which are one species of conservatively modified variations. Every nucleic acid sequence herein which encodes a polypeptide also describes silent variations of the nucleic acid. One of skill will recognize that in certain contexts each codon in a nucleic acid (except AUG, which is ordinarily the only codon for methionine, and TGG, which is ordinarily the only codon for tryptophan) can be modified to yield a functionally identical molecule. Accordingly, often silent variations of a nucleic acid which encodes a polypeptide is implicit in a described sequence with respect to the expression product, but not with respect to actual probe sequences.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention. Typically conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

“Homologous,” in relation to two or more peptides, refers to two or more sequences or subsequences that have a specified percentage of amino acid residues that are the same (i.e., about 60% identity, preferably 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site http://www.ncbi.nlm.nih.gov/BLAST/or the like). The definition also includes sequences that have deletions and/or additions, as well as those that have substitutions, as well as naturally occurring, e.g., polymorphic or allelic variants, and man-made variants. As described below, the preferred algorithms can account for gaps and the like. Preferably, identity exists over a region that is at least about 25 amino acids in length, or more preferably over a region that is 50-100 amino acids in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Preferably, default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of one of the number of contiguous positions selected from the group consisting typically of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

Preferred examples of algorithms that are suitable for determining percent sequence identity and sequence similarity include the BLAST and BLAST 2.0 algorithms, which are described in Altschul et al., Nuc. Acids Res. 25:3389-3402 (1977) and Altschul et al., J. Mol. Biol. 215:403-410 (1990). BLAST and BLAST 2.0 are used, with the parameters described herein, to determine percent sequence identity for the nucleic acids and proteins of the invention. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, e.g., for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a peptide is considered similar to a reference sequence if the smallest sum probability in a comparison of the test peptide to the reference peptide is less than about 0.2, more preferably less than about 0.01, and most preferably less than about 0.001. Log values may be large negative numbers, e.g., 5, 10, 20, 30, 40, 40, 70, 90, 110, 150, 170, etc.

A “label” or a “detectable moiety” is a composition detectable by spectroscopic, photochemical, biochemical, immunochemical, chemical, or other physical means. For example, useful labels include fluorescent dyes, electron-dense reagents, enzymes (e.g., as commonly used in an ELISA), biotin, digoxigenin, or haptens and proteins or other entities which can be made detectable, e.g., by incorporating a radiolabel into the peptide or used to detect antibodies specifically reactive with the peptide. The radioisotope may be, for example, 3H, 14C, 32P, 35S, or 125I. In some cases, particularly using anti-α5β1 integrin antibodies, the radioisotopes are used as toxic moieties, as described below. The labels may be incorporated into the antibodies at any position. Any method known in the art for conjugating the antibody to the label may be employed, including those methods described by Hunter et al., Nature, 144:945 (1962); David et al., Biochemistry, 13:1014 (1974); Pain et al., J. Immunol. Meth., 40:219 (1981); and Nygren, J. Histochem. and Cytochem., 30:407 (1982). The lifetime of radiolabeled peptides or radiolabeled antibody compositions may extended by the addition of substances that stablize the radiolabeled peptide or antibody and protect it from degradation. Any substance or combination of substances that stablize the radiolabeled peptide or antibody may be used including those substances disclosed in U.S. Pat. No. 5,961,955.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, e.g., recombinant cells express genes that are not found within the native (non-recombinant) form of the cell or express native genes that are otherwise abnormally expressed, under expressed or not expressed at all. By the term “recombinant nucleic acid” herein is meant nucleic acid, originally formed in vitro, in general, by the manipulation of nucleic acid, e.g., using polymerases and endonucleases, in a form not normally found in nature. In this manner, operably linkage of different sequences is achieved. Thus an isolated nucleic acid, in a linear form, or an expression vector formed in vitro by ligating DNA molecules that are not normally joined, are both considered recombinant for the purposes of this invention. It is understood that once a recombinant nucleic acid is made and reintroduced into a host cell or organism, it will replicate non-recombinantly, i.e., using the in vivo cellular machinery of the host cell rather than in vitro manipulations; however, such nucleic acids, once produced recombinantly, although subsequently replicated non-recombinantly, are still considered recombinant for the purposes of the invention. Similarly, a “recombinant protein” is a protein made using recombinant techniques, i.e., through the expression of a recombinant nucleic acid as depicted above.

“Eye tissue” refers to any tissue type, or combination of tissue types, found in a vertebrate eye. Examples of eye tissue include retinal, vitreal, macular and corneal tissue. “Affected eye” refers to the eye having wound tissue responsive to the granulation inhibitors of the present invention.

“Injured or diseased tissue” or “wound tissue” refer to any tissue that has been subjected to a trauma sufficient to induce cellular granulation. “Wound site” refers to the region of wound tissue at which cellular granulation occurs. Trauma sufficient to induce granulation can result from physical, chemical or infectious invasion of the affected tissue. Trauma can be created by abnormal physiological events, such as an auto immune response, or by pathogen invasion for example by fungi or bacteria.

“Lesions” refers to a localized area of tissue damage created by trauma resulting from physical, chemical or infectious insult to the tissue. In the context of the present invention, lesions create a wound site and resulting granulation.

“Infection” refers to an invasion by and multiplication of pathogenic microorganisms in a bodily part or tissue. In the context of the present invention, an infection of a tissue produces subsequent tissue injury, resulting in wound tissue.

“Granulation” or “cellular granulation” refers to that part of the wound healing process where small, red, grainlike prominences form on the raw surface of a lesion or wound site, generally promoting the process of healing. In some cases however, granulation can be excessive resulting in compromising the healed tissue unnecessarily or causing damage to surrounding tissue(s). “Reducing granulation” is the process where excessive granulation is controlled or eliminated, thereby minimizing healed tissue that is weakened, or damage to surrounding tissue resulting from excessive granulation.

Deleterious granulation refers to granulation that occurs after the initial wound and causes wounding of tissue beyond the original wound site. Deleterious granulation is generally the result of the anatomical environment in which the wound site occurs. Exemplary tissues where wound sites would be subject to deleterious granulation include those at or near a surface bounding a lumen, such as the macula of the eye (the vitreal space) joint tissue (synovial space), and alveolar membranes (alveolar space).

“Macrophage behavior” refers to a phenotypic behavior of non-macrophage cell types that mimics the behavior of activated macrophages. Exemplary macrophage behavior includes phagocytic activity directed toward cellular and foreign debris or infectious agents such as bacteria, and secretion of growth and paracrine factors such as cytokines, chemokines or mediators of inflammatory responses.

“RPE cell behavior” refers to the phenotypic activity of retinal pigment epithelial cells that form a cell layer beneath the retina and support the function of photoreceptor cells. Photoreceptor cells depend on the RPE to provide nutrients and eliminate waste products. RPE cell behavior includes the response of RPE cells to lesions forming wound tissue in an affected eye. In response to a lesion, RPE cells appear to transform taking on macrophage behavior. Granulation inhibitors of the present invention alter this RPE cell behavior to wounding by directing the cells to take on a fibroblast-like morphology instead of macrophage behavior.

“Stained tissue sections” refers to thin slices of tissue that have been impregnated with one or more dyes or labels that aid in identifying features present in the slice of tissue. Tissue staining kits are well known by those of skill in the art and are commercially available, for example from SANYO Gallenkamp plc, Monarch Way, Belton Park, Loughborough, Leicestershire LE11 5XG.

Granulation inhibitors, binding agents, inhibitor and binding candidates, and compositions containing these compounds can be applied to a wound tissue in a variety of ways. As used herein, “direct application” refers to contacting the compound directly to the wound site. “Inravitreal injection” refers to injecting the compound into the vitreous humor of the eye and allowing the compound to diffuse to the wound site, or be carried to a wound site through the affected subjects vascular system. “Scleral injection” refers to injection of the granulation inhibitor directly into the sclera of the eye. “Systemic injection” refers to injection at a site distant from the wound site to be treated. Systemic injection includes intravenous, subcutaneous and intramuscular injection. “Nebulized inhalation” refers to dispersing the liquefied compound in fine droplets, which are then inhaled. Nebulized inhalation is particularly useful for treatment of wound site(s) in the lungs, or the compound can be absorbed in the aveoli and transported to a distant wound site via the vascular system. “Eye drop” refers to the application of a liquefied compound to the external surface of the eye of an affected individual.

II. Introduction

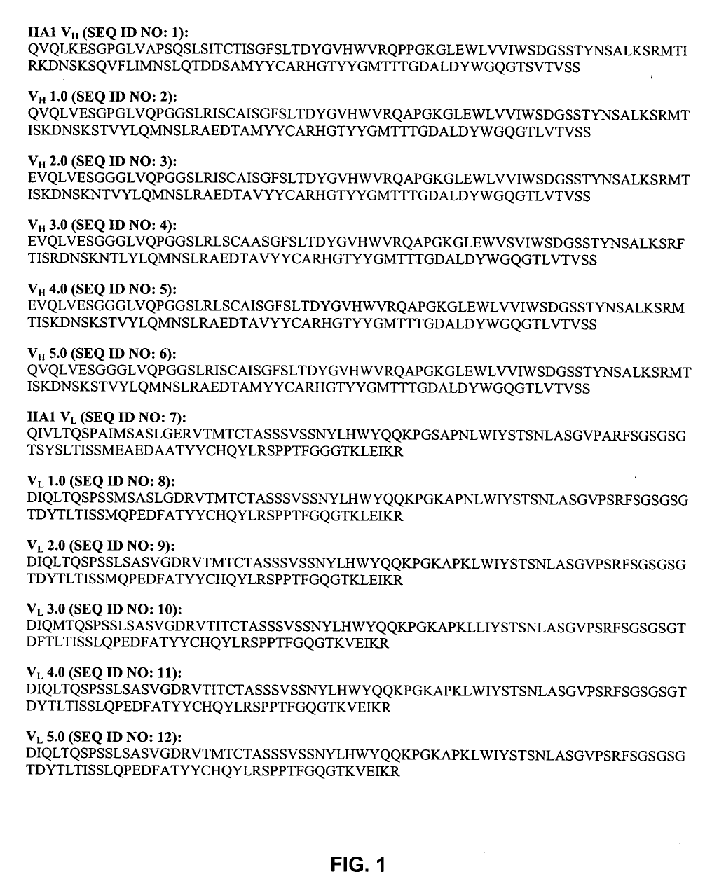

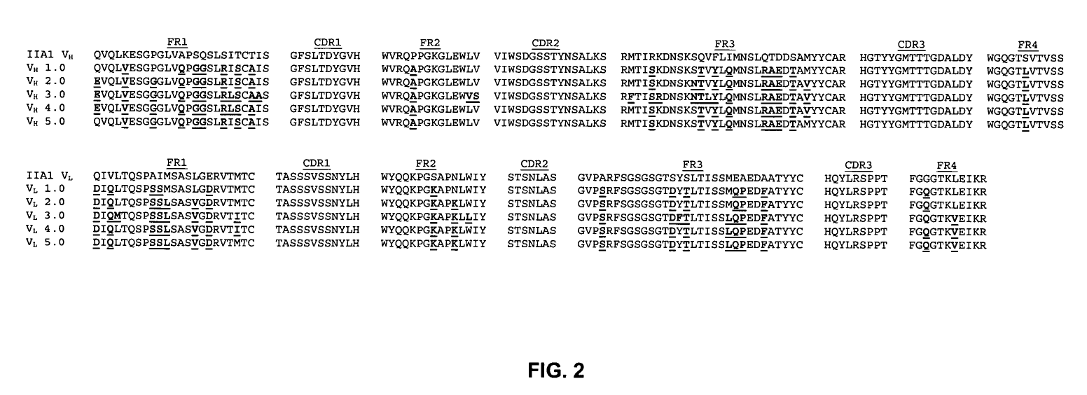

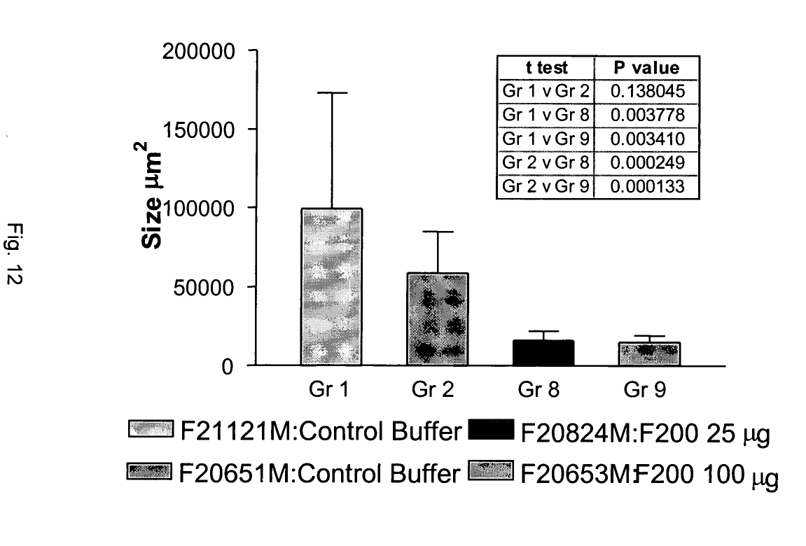

The present invention provides methods that enable the user to identify inhibitors of tissue granulation in and around a wound site, thereby limiting excessive scar formation as the wounded tissue heals. The efficacy of the present methods is illustrated in FIG. 12, which shows an almost five fold reduction of scar tissue formation at the site of retinal injuries treated by intravitreal injection of 25 μkg or 100 μg of the granulation inhibitor EOS200F, when compared to control subjects treated with a buffer solution. Preferred granulation inhibitors of the present invention are antibodies that bind competitively for a5b 1 integrin with antibody having a variable heavy chain region having an amino acid sequence homologous to an amino acid sequence selected from the group consisting of SEQ ID NOS.: 1-6, and a variable light chain region having an amino acid sequence homologous to an amino acid sequence selected from the group consisting of SEQ ID NOS.: 7-12; more preferably granulation inhibitors are antibodies having a variable heavy chain region having an amino acid sequence homologous to an amino acid sequence selected from the group consisting of SEQ ID NOS.: 1-6 (see also FIGS. 1 and 2), and a variable light chain region having an amino acid sequence homologous to an amino acid sequence selected from the group consisting of SEQ ID NOS.: 7-12 (see also FIGS. 1 and 2).

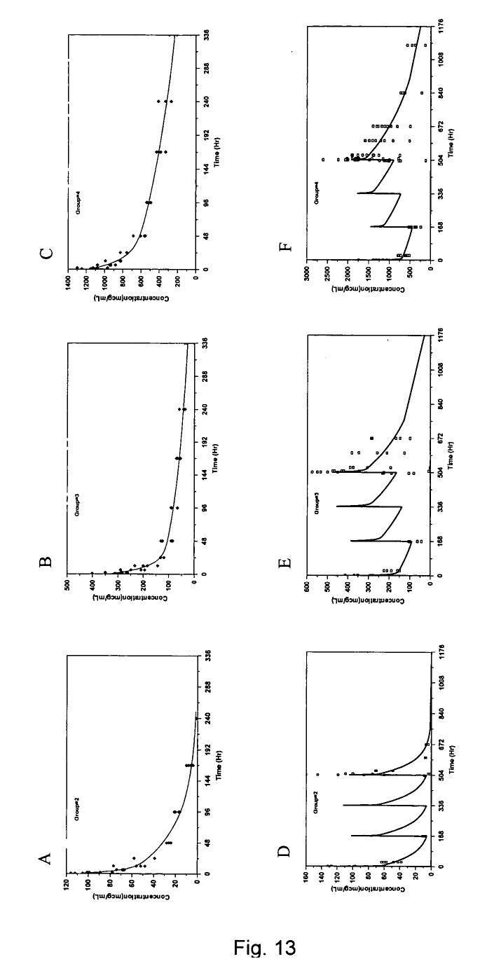

Granulation inhibitors of the present invention can be delivered locally or systemically by a variety of techniques as described herein. FIG. 13 illustrates the serum levels of the granulation inhibitor M200 (formerly referred to as “EOS200-4”) at different times after initial intravenous doses of 5 mg/kg (FIG. 13A), 15 mg/kg (FIG. 13B), and 50 mg/kg (FIG. 13C). Briefly, individuals were injected intravenously with 5 mg/kg, 15 mg/kg, or 50 mg/kg and sera collected and tested for M200 for each dose level on the days indicated.

FIGS. 13D-13F illustrate that therapeutic dose levels can be maintained in an individual by weekly intravenous dosing. Briefly, each week individuals were injected intravenously with 5 mg/kg, 15 mg/kg, or 50 mg/kg and sera collected and tested for M200 for each dose level on the days indicated. Therapeutic levels of granulation inhibitor were maintained at least for the 15 and 50 mg/kg dosings.

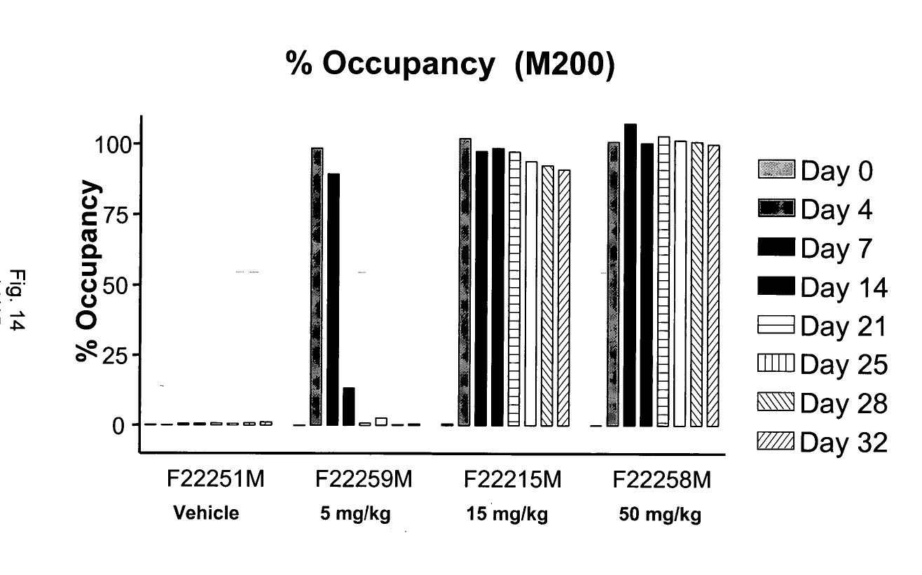

FIG. 14 illustrates monocyte α5β1 integrin binding of the granulation inhibitor M200, confirming that the inhibitor is not functionally degraded and remains active in sera. Briefly, individuals where given single doses at the indicated amounts as described previously. FACS studies were conducted on whole blood monocytes collected on the days indicated. As can be seen, M200 occupancy of binding sites associated with monocytes α5β1 integrin correlates with serum levels of the granulation inhibitor for each day. From these studies, it can be determined that approximately 60 μg/ml sera granulation inhibitor is sufficient to completely saturate blood monocytes α5β1 integrin binding sites.

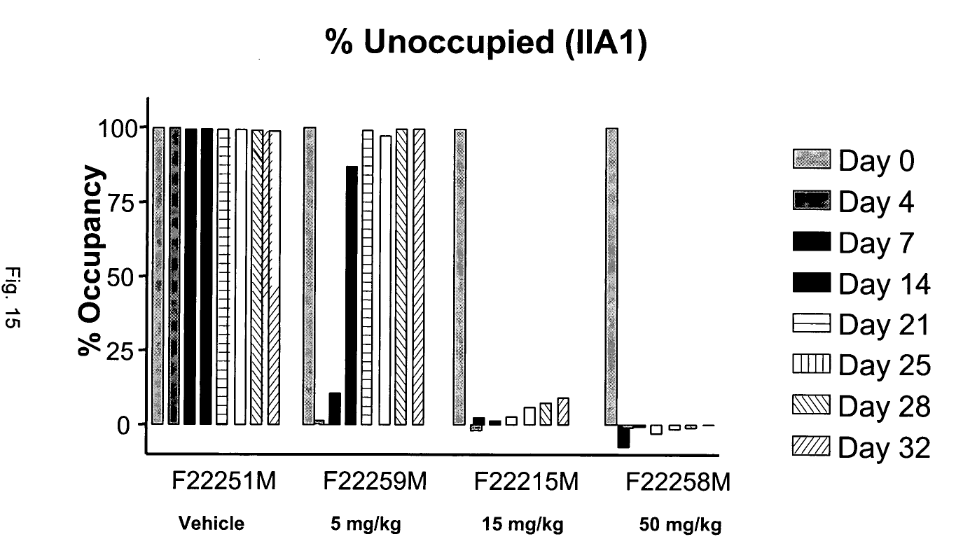

FIG. 15 confirms the result of FIG. 14. FIG. 15 is a competitive FACS assay using a monocytes α5β1 integrin in an inverse relation to M200 binding, and is completely blocked at those data points where M200 is saturating.

As many of the granulation inhibitors identified by the methods of the present invention are able to permeate capillary membranes and/or basal membrane layers, these inhibitors may be applied topically, in addition to systemic and direct application.

III. Preparation of α5β1 Integrin Binding Agent and Granulation Inhibitor Compounds and Libraries

As disclosed herein, proteins, particularly antibodies, muteins, nucleic acid aptamers, and peptide and nonpeptide small organic molecules that bind of α5β1 integrin may serve as binding agents and granulation inhibitors of the present invention. Binding agents may be isolated from natural sources, prepared synthetically or recombinantly, or any combination of the same.

For example, peptides may be produced synthetically using solid phase techniques such as described in “Solid Phase Peptide Synthesis” by G. Barany and R. B. Merrifield in Peptides, Vol. 2, edited by E. Gross and J. Meienhoffer, Academic Press, New York, N.Y., pp. 100-118 (1980). Similarly, nucleic acids can also be synthesized using the solid phase techniques, such as those described in Beaucage, S. L., & Iyer, R. P. (1992) Advances in the synthesis of oligonucleotides by the phosphoramidite approach. Tetrahedron, 48, 2223-2311; and Matthes et al., EMBO J., 3:801-805 (1984).

Modifications of peptides of the present invention with various amino acid mimetics or unnatural amino acids are particularly useful in increasing the stability of the peptide in vivo. Stability can be assayed in a number of ways. For instance, peptidases and various biological media, such as human plasma and serum, have been used to test stability. See, e.g., Verhoef et al., Eur. J. Drug Metab Pharmacokin. 11:291-302 (1986). Half life of the peptides of the present invention is conveniently determined using a 25% human serum (v/v) assay. The protocol is generally as follows. Pooled human serum (Type AB, non-heat inactivated) is delipidated by centrifugation before use. The serum is then diluted to 25% with RPMI tissue culture media and used to test peptide stability. At predetermined time intervals a small amount of reaction solution is removed and added to either 6% aqueous trichloracetic acid or ethanol. The cloudy reaction sample is cooled (4° C.) for 15 minutes and then spun to pellet the precipitated serum proteins. The presence of the peptides is then determined by reversed-phase HPLC using stability-specific chromatography conditions. Other useful peptide modifications known in the art include glycosylation and acetylation.

In the case of nucleic acids, existing sequences can be modified using recombinant DNA techniques well known in the art. For example, single base alterations can be made using site-directed mutagenesis techniques, such as those described in Adelman et al., DNA, 2:183, (1983).

Alternatively, nucleic acids can be amplified using PCR techniques or expression in suitable hosts (cf. Sambrook et al., Molecular Cloning: A Laboratory Manual, 1989, Cold Spring Harbor Laboratory, New York, USA). Peptides and proteins may be expressed using recombinant techniques well known in the art, e.g., by transforming suitable host cells with recombinant DNA constructs as described in Morrison, J. Bact., 132:349-351 (1977); and Clark-Curtiss & Curtiss, Methods in Enzymology, 101:347-362 (Wu et al., eds, 1983).

Peptides and nucleic acids of the present invention may also be available commercially, or may be produced commercially, given the structural and/or functional properties of the molecules desired.

The present invention also contemplates α5 β1 integrin binding agents that are nonpeptide, small organic molecules including a peptidomimetic, which is an organic molecule that mimics the structure of a peptide; or a peptoid such as a vinylogous peptoid. A nonpeptide small organic molecule that may act as an α5β1 integrin binding agent and granulation inhibitor could be, for example, a heterocycle having the general structure (S)-2-phenylsulfonylamino-3-{{{8-(2-pyridinyl aminomethyl)-}-1-oxa-2-azas-piro-{4,5}-dec-2-en-yl}carbonylamino}propionic acid; (S)-2-{(2,4,6-trimethylphenyl)sulfonyl}amino-3-{7-benzyloxycarbonyl-8-(2--pyridinyl aminomethyl)-1-oxa-2,7-diazaspiro-{4,4}-non-2-en-3-yl}carbonylamino}propionic acid (see U.S. Pat. No. 5,760,029). Additional nonpeptide, small organic molecule α5β1 binding agents useful in a method of the invention can be identified by screening, for example, chemically modified derivatives of a heterocycle having the structure disclosed above, or other libraries of nonpeptide, small organic molecules (see below).

Prefered embodiments of the present invention include granulation inhibitors that are α5β1 antibodies, preferably chimeric, most preferably humanized antibodies. Methods for producing such antibodies are discussed immediately below.

A. Antibody Granulation Inhibitors

Anti-integrin antibodies, including anti-α5β1 integrin antibodies, can be purchased from a commercial source, for example, Chemicon, Inc. (Temecula Calif.), or can be raised using as an immunogen, such as a substantially purified full length integrin, which can be a human integrin, mouse integrin or other mammalian or nonmammalian integrin that is prepared from natural sources or produced recombinantly, or a peptide portion of an integrin, which can include a portion of the RGD binding domain, for example, a synthetic peptide. A non-immunogenic peptide portion of an integrin such as a human a5b1 can be made immunogenic by coupling the hapten to a carrier molecule such bovine serum albumin (BSA) or keyhole limpet hemocyanin (KLH), or by expressing the peptide portion as a fusion protein. Various other carrier molecules and methods for coupling a hapten to a carrier molecule are well known in the art and described, for example, by Harlow and Lane (supra, 1988).

Particularly useful antibodies for performing methods of the invention are humanized antibodies that that specifically bind to α5β1 integrin. Such antibodies are particularly useful where they bind α5β1 integrin with at least an order of magnitude greater affinity than they bind another integrin, for example, αVβ3 or αVβ5. Methods for creating chimeric antibodies, including humanized antibodies, is discussed in greater detail below.

1. Production of Recombinant Antibody Granulation Inhibitors

In order to prepare recombinant chimeric and humanized antibodies that may function as granulation inhibitors of the present invention, the nucleic acid encoding non-human antibodies must first be isolated. This is typically done by immunizing an animal, for example a mouse, with prepared α5β1 integrin or an antigenic peptide derived therefrom. Typically mice are immunized twice intraperitoneally with approximately 50 micrograms of protein antibody per mouse. Sera from immunized mice can be tested for antibody activity by immunohistology or immunocytology on any host system expressing such polypeptide and by ELISA with the expressed polypeptide. For immunohistology, active antibodies of the present invention can be identified using a biotin-conjugated anti-mouse immunoglobulin followed by avidin-peroxidase and a chromogenic peroxidase substrate. Preparations of such reagents are commercially available; for example, from Zymad Corp., San Francisco, Calif. Mice whose sera contain detectable active antibodies according to the invention can be sacrificed three days later and their spleens removed for fusion and hybridoma production. Positive supernatants of such hybridomas can be identified using the assays common to those of skill in the art, for example, Western blot analysis.

The nucleic acids encoding the desired antibody chains can then be isolated by, for example, using hybridoma mRNA or splenic mRNA as a template for PCR amplification of the heavy and light chain genes [Huse, et al., Science 246:1276 (1989)]. Nucleic acids for producing both antibodies and intrabodies can be derived from murine monoclonal hybridomas using this technique [Richardson J. H., et al., Proc Natl Acad Sci USA 92:3137-3141 (1995); Biocca S., et al., Biochem and Biophys Res Comm, 197:422-427 (1993) Mhashilkar, A. M., et al., EMBO J 14:1542-1551 (1995)]. These hybridomas provide a reliable source of well-characterized reagents for the construction of antibodies and are particularly useful once their epitope reactivity and affinity has been characterized. Isolation of nucleic acids from isolated cells is discussed further in Clackson, T., et al., Nature 352:624-628 (1991) (spleen) and Portolano, S., et al., supra; Barbas, C. F., et al., supra; Marks, J. D., et al., supra; Barbas, C. F., et al., Proc Natl Acad Sci USA 88:.7978-7982 (1991) (human peripheral blood lymphocytes). Humanized antibodies optimally include at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

A number of methods have been described to produce recombinant antibodies, both chimeric and humanized. Controlled rearrangement of antibody domains joined through protein disulfide bonds to form chimeric antibodies may be utilized (Konieczny et al., Haematologia, 14(1):95-99, 1981). Recombinant DNA technology can also be used to construct gene fusions between DNA sequences encoding mouse antibody variable light and heavy chain domains and human antibody light and heavy chain constant domains (Morrison et al., Proc. Natl. Acad. Sci. USA, 81(21):6851-6855, 1984.).

DNA sequences encoding the antigen binding portions or complementarity determining regions (CDR's) of murine monoclonal antibodies may be grafted by molecular means into the DNA sequences encoding the frameworks of human antibody heavy and light chains (Jones et al., Nature, 321(6069):522-525, 1986.; Riechmann et al., Nature, 332(6162):323-327, 1988.). The expressed recombinant products are called “reshaped” or humanized antibodies, and comprise the framework of a human antibody light or heavy chain and the antigen recognition portions, CDR's, of a murine monoclonal antibody.

Other methods for producing humanized antibodies are described in U.S. Pat. Nos. 5,693,762; 5,693,761; 5,585,089; 5,639,641; 5,565,332; 5,733,743; 5,750,078; 5,502,167; 5,705,154; 5,770,403; 5,698,417; 5,693,493; 5,558,864; 4,935,496; 4,816,567; and 5,530,101, each incorporated herein by reference.

Techniques described for the production of single chain antibodies (U.S. Pat. No. 4,946,778) can be adapted to produce single chain humanized antibodies to α5β1 integrin.

2. Isolation of Antibody Granulation Inhibitors

Affinity Purification

Affinity purification of an antibody pool or sera provides a practitioner with a more uniform reagent. Methods for enriching antibody granulation inhibitors using antibody affinity matrices to form an affinity column are well known in the art and available commercially (AntibodyShop, c/o Statens Serum Institut, Artillerivej 5, Bldg. P2, DK-2300 Copenhagen S). Briefly, an antibody affinity matrix is attached to an affinity support (see e.g.; CNBR Sepharose (R), Pharmacia Biotech). A mixture comprising antibodies is then passed over the affinity matrix, to which the antibodies bind. Bound antibodies are released by techniques common to those familiar with the art, yielding a concentrated antibody pool. The enriched antibody pool can then be used for further immunological studies, some of which are described herein by way of example. Although the antibody affinity matrices used to isolate the antibodies of the present invention are not designed to specifically recognize the anti-α5β1 integrin antibodies of the present invention, this does not limit the utility of the affinity matrices in purifying the antibodies, as the antibodies are expressed as recombinant proteins in systems that are monoclonal in their nature.

pH-sensitive Antibody Purification

Some antibody binding agents of the present invention display a propensity to precipitate when affinity purified at neutral or basic pH. To address this issue, a process for purification of pH-sensitive antibodies, including the antibodies indicated in FIG. 1, and chimeric antibodies that include the mouse variable region or have 80% or more sequence identity with the mouse variable region, or having 80% or more sequence identity to the CDR regions of the antibodies included in FIG. 1 has been devised. The process includes conducting affinity chromatography for the antibody using a chromatographic column, e.g. an ion exchange column, that contains bound Antibody affinity matrix, followed by eluting the antibody at a pH of from about 3.0 to about 5.5, preferably from about 3.3 to about 5.5, and most preferably either from about 3.5 to about 4.2 or from about 4.2 to about 5.5. Lower pH values within this range are more suitable for small-scale purification while a pH of about 4.2 or higher is considered more suitable for larger scale operations. Operation of the purification process within this range produces a product with little or no aggregation, most preferably with essentially no aggregation.

Affinity chromatography is one means known in the art for isolating or purifying a substance, such as an antibody or other biologically active macromolecule. This is accomplished in general by passing a solution containing the antibody through a chromatographic column that contains one or more ligands that specifically bind to the antibody immobilized on the column. Such groups can extract the antibody from the solution through ligand-affinity reactions. Once that is accomplished, the antibody may be recovered by elution from the column.

The purification process involves the absorption of the antibodies onto antibody affinity matrix bound to a substrate. Various forms of antibody affinity matrix may be used. The only requirement is that the antibody affinity matrix molecule possesses the ability to bind the antibody that is to be purified. For example, antibody affinity matrix isolated from natural sources, antibody affinity matrix produced by recombinant DNA techniques, modified forms of antibody affinity matrices, or fragments of these materials which retain binding ability for the antibody in question may be employed. Exemplary materials for use as antibody affinity matrices include polypeptides, polysaccharides, fatty acids, lipids, nucleic acid aptamers, glycoproteins, lipoproteins, glycolipids, multiprotein complexes, a biological membrane, viruses, protein A, protein G, lectins, and Fc receptors.

The antibody affinity matrix is attached to a solid phase or support by a general interaction (for example, by non-specific, ion exchange bonding, by hydrophobic/hydrophilic interactions), or by a specific interaction (for example, antigen-antibody interaction), or by covalent bonding between the ligand and the solid phase, or other methods known by those of skill in the art. Alternately, an intermediate compound or spacer can be attached to the solid phase and the antibody affinity matrix can then be immobilized on the solid phase by attaching the affinity matrix to the spacer. The spacer can itself be a ligand (i.e., a second ligand) that has a specific binding affinity for the free antibody affinity matrix.

The antibodies may be eluted from the substrate-bound antibody affinity matrix using conventional procedures, e.g. eluting the antibodies from the column using a buffer solution. To minimize precipitation, pH-sensitive anti-α5β1 integrin antibodies are preferably eluted with a buffer solution comprising 0.1 M glycine at pH 3.5. To minimize degradation and/or denaturation, the temperature of the buffer solution is preferably kept below 10° C., more preferably at or below 4° C. For the same reasons, the period during which the antibodies are exposed to acidic pH should also be minimized. This is accomplished, for example, by adding a predetermined amount of a basic solution to the eluted antibody solution. Preferably this basic solution is a buffered solution, more preferably a volatile basic buffered solution, most preferably an ammonia solution.

The elution of antibodies from the substrate-bound antibody affinity matrix may be monitored by various methods well-known in the art. For example, if column procedures are employed, fractions may be collected from the columns, and the presence of protein determined by measuring the absorption of the fractions. If antibodies of known specificity are being purified, the presence of the antibodies in fractions collected from the columns may be measured by immunoassay techniques, for example, radioimmunoassay (RIA) or enzyme immunoassay (EIA).

The process of the present invention may be performed at any convenient temperature which does not substantially degrade the antibody being purified, or detrimentally affect the antibody affinity matrix bound to a substrate Preferably, the temperature employed is room temperature. The antibodies eluted from the antibody affinity matrix column may be recovered, if desired, using various methods known in the art.

B. Small Molecule Granulation Inhibitors

A combinatorial chemical library is a collection of diverse chemical compounds generated by either chemical synthesis or biological synthesis, by combining a number of chemical “building blocks” such as reagents. For example, a linear combinatorial chemical library such as a polypeptide library is formed by combining a set of chemical building blocks (amino acids) in every possible way for a given compound length (i.e., the number of amino acids in a polypeptide compound). Millions of chemical compounds can be synthesized through such combinatorial mixing of chemical building blocks.

Preparation and screening of combinatorial chemical libraries is well known to those of skill in the art. Such combinatorial chemical libraries include, but are not limited to, peptide libraries (see, e.g., U.S. Pat. No. 5,010,175, Furka, Int. J. Pept. Prot. Res. 37:487-493 (1991) and Houghton et al., Nature 354:84-88 (1991)). Other chemistries for generating chemical diversity libraries can also be used. Such chemistries include, but are not limited to: peptides (e.g., PCT Publication No. WO 91/19735), encoded peptides (e.g., PCT Publication WO 93/20242), random bio-oligomers (e.g., PCT Publication No. WO 92/00091), benzodiazepines (e.g., U.S. Pat. No. 5,288,514), diversomers such as hydantoins, benzodiazepines and dipeptides (Hobbs et al., Proc. Nat. Acad. Sci. USA 90:6909-6913 (1993)), vinylogous polypeptides (Hagihara et al., J. Amer. Chem. Soc. 114:6568 (1992)), nonpeptidal peptidomimetics with glucose scaffolding (Hirschmann et al., J. Amer. Chem. Soc. 114:9217-9218 (1992)), analogous organic syntheses of small compound libraries (Chen et al., J. Amer. Chem. Soc. 116:2661 (1994)), oligocarbamates (Cho et al., Science 261:1303 (1993)), and/or peptidyl phosphonates (Campbell et al., J. Org. Chem. 59:658 (1994)), nucleic acid libraries (see Ausubel, Berger and Sambrook, all supra), peptide nucleic acid libraries (see, e.g., U.S. Pat. No. 5,539,083), antibody libraries (see, e.g., Vaughn et al., Nature Biotechnology, 14(3):309-314 (1996) and PCT/US96/10287), carbohydrate libraries (see, e.g., Liang et al., Science, 274:1520-1522 (1996) and U.S. Pat. No. 5,593,853), small organic molecule libraries (see, e.g., benzodiazepines, Baum C&EN, Jan. 18, page 33 (1993); isoprenoids, U.S. Pat. No. 5,569,588; thiazolidinones and metathiazanones, U.S. Pat. No. 5,549,974; pyrrolidines, U.S. Pat. Nos. 5,525,735 and 5,519,134; morpholino compounds, U.S. Pat. No. 5,506,337; benzodiazepines, 5,288,514, and the like).

Another approach uses recombinant bacteriophage to produce large libraries. Using the “phage method” (Scott and Smith, Science 249:386-390, 1990; Cwirla, et al, Proc. Natl. Acad. Sci., 87:6378-6382, 1990; Devlin et al., Science, 49:404-406, 1990), very large libraries can be constructed (10⁶-10⁸ chemical entities). A second approach uses primarily chemical methods, of which the Geysen method (Geysen et al., Molecular Immunology 23:709-715, 1986; Geysen et al. J. Immunologic Method 102:259-274, 1987; and the method of Fodor et al. (Science 251:767-773, 1991) are examples. Furka et al. (14th International Congress of Biochemistry, Volume #5, Abstract FR:013, 1988; Furka, Int. J. Peptide Protein Res. 37:487-493, 1991), Houghton (U.S. Pat. No. 4,631,211, issued December 1986) and Rutter et al. (U.S. Pat. No. 5,010,175, issued Apr. 23, 1991) describe methods to produce a mixture of peptides that can be tested as agonists or antagonists.

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.). In addition, numerous combinatorial libraries are themselves commercially available (see, e.g., ComGenex, Princeton, N.J., Tripos, Inc., St. Louis, Mo., 3D Pharmaceuticals, Exton, Pa., Martek Biosciences, Columbia, Md., etc.).

Small peptides suitable for use as granulation inhibitors are discussed in Horton M. “Arg-gly-Asp (RGD) peptides and peptidomimetics as therapeutics: relevance for renal diseases.”Exp Nephrol. 1999 Mar.-Apr.;7(2):178-84; Pasqualini R, Koivunen E, Ruoslahti E. “A peptide isolated from phage display libraries is a structural and functional mimic of an RGD-binding site on integrins” J Cell Biol. 1995 Sep.;130(5):1189-96; Koivunen E, Wang B, Ruoslahti E. “Isolation of a highly specific ligand for the alpha 5 beta 1 integrin from a phage display library.” J Cell Biol. February 1994; 124(3):373-80; U.S. Pat. No. 6,177,542 and related patents to Ruoslahti, et al.

Small double-stranded RNAs, or siRNAs, are also contemplated by the present invention. siRNAs of the invention have a sequence identical to the sequence of one of the α5β1 integrin subunits. When applied to a cell expressing α5β1 integrin, these siRNAs inhibit translation of the α5β1 integrin subunit having the siRNA sequence by causing the degradation of the corresponding mRNA transcript encoding the subunit.

C. General Methods for Isolating Granulation Inhibitors

Methods for isolating granulation inhibitors are well known in the art. Generally any purification protocol suitable for isolating nucleic acids or proteins can be used. For example, affinity purification as discussed above in the context of antibody granulation inhibitor isolation can be used in a more general sense to isolate any α5β1 integrin-binding granulation inhibitor. Nucleic acid granulation inhibitors can be also be purified using agarose gel electrophoresis, as is known in the art. Column chromatography techniques, precipitation protocols and other methods for separating proteins and/or nucleic acids may also be used. (see, e.g., Scopes, Protein Purification: Principles and Practice (1982); U.S. Pat. No. 4,673,641; Ausubel et al., supra; and Sambrook et al., supra; and Leonard et al., J. Biol. Chem. 265:10373-10382 (1990).

IV. Methods for Identifying Granulation Inhibitors

The present invention provides methods for identifying diagnostic and therapeutic granulation inhibitors. An exemplary method for identifying granulation inhibitors involves evaluating the effects of inhibitor candidates on the formation of granulation or scar tissue at a wound sites created under controlled conditions. Similar wound sites are first formed in the same living tissue of two different subjects. Wound sites can be formed using any suitable method, such as surgical puncture, cutting, burning, for example with a laser, or chemical irritation and the like. Suitable tissues for screening assays may include eye, skin, bone, cartilage, vascular, ligament and tendon.

Once the wound sites are formed, one wound site (test wound site) is treated with a predetermined dose of a granulation inhibitor candidate. The second wound site (control site) is treated with a control solution, preferably a non-irritating buffer solution or other carrier.

When the granulation inhibitor candidate is delivered in a carrier, the control solution is ideally the carrier absent the granulation inhibitor candidate. Test and control wound sites should be in different individuals or separate tissue samples, as many granulation inhibitors currently known can cross capillary membranes. If the control and test wound sites are placed respectively, for example, in the right and left eyes of the same individual, granulation inhibitor applied to the test site can reach the control site through the individuals vascular system, leading to aberrant results. Multiple doses of the granulation inhibitor candidate may be applied to the test wound site, preferably following a predetermined schedule of dosing. The dosing schedule may be over a period of days, more preferably over a period of weeks.

Once the dosing schedule has been completed, both test and control wound sites are examined to determine the level of granulation or scarring that is present. This may be accomplished by any suitable method, for example by making tissue sections that are suitable for staining and microscopic examination (granulation), or simply microscopic examination (scar tissue). Methods for performing microscopic examination and tissue sectioning, staining are well known in the art. A granulation inhibitor candidate suitable for use as a granulation inhibitor is identified by noting significantly reduced granulation in tissue sections taken from the test site when compared to the control site. Ideally granulation or scarring at the test wound site should be at least 75%, more preferably 50%, most preferably 30% or less granulation than is present in the control wound site. Where necessary, levels of granulation or scarring may be calculated by determining the area of granulation tissue present at each wound site. Calculations may be performed by constructing a 2-dimensional image of the granulation tissue at each wound site and calculating the area held within the image. Such calculations are preferably performed with the aid of a digital computer, ideally a digital computer linked to a microscope. Scar tissue may be quantified by determining the surface area covered by the scar.

In an exemplary embodiment, wound sites are induced by laser treatment to the maculae of the eyes of two primates. Other eye tissues may optionally be used, for example, retinal or corneal tissue. The wound site in each eye should ideally be placed in a similar location relative to proximate anatomical features. The size of each wound should be similar, preferably about 25 μm, more preferably about 50 μm, advantageously about 100 μm, more advantageously about 200 μm in diameter. Laser settings ideally should be just sufficient to create a wound site that induce granulation, i.e., preferably about 200 milliwatts, more preferably about 300 milliwatts, advantageously about 450 milliwatts, more advantageously about 500 milliwatts, ideally between about 300 milliwatts and about 700 milliwatts. Apparatus for inducing such wound sites are commercieally available, e.g., OcuLight GL (532 nm) Laser Photo-coagulator with a IRIS Medical® Portable Slit Lamp Adaptor].

Intravitreal injection of a granulation inhibitor candidate, for example an antibody having a variable heavy chain region having an amino acid sequence homologous to an amino acid sequence selected from the group consisting of SEQ ID NOS.: 1-6, and a variable light chain region having an amino acid sequence homologous to an amino acid sequence selected from the group consisting of SEQ ID NOS.: 7-12, is then performed in each eye.

The first injection may be made immediately following laser treatment. The needle of the dose syringe would be passed through the sclera and pars plana to a position approximately 4 mm posterior to the limbus. The needle should be directed posterior to the lens into the mid-vitreous and slowly injected into the vitreous. Identical dosing should be done on a weekly basis for four weeks. Suitable dosage will depend on the nature of the particular granulation inhibitor candidate being tested. By way of example, Fab fragments should be given at a dose of about 25 μg, preferably about 50 μg, more preferably about 100 μg most preferably about 200 μg per eye, assuming a vitreal volume of 2 ml. As a baseline for determining dosages of other inhibitor candidates, this corresponds to approximately 1 μM granulation inhibitor. Using this baseline value, one of skill in the art can determine dose levels for other granulation inhibitor candidates.

In dosing it should be noted that systemic injection, either intravenously, subcutaneously or intramuscularly, may also be used. For systemic injection of a granulation inhibitor or a granulation inhibitor candidate dosage should be about 5 mg/kg, preferably more preferably about 15 mg/kg, advantageously about 50 mg/kg, more advantageously about 100 mg/kg, acceptably about 200 mg/kg. dosing performed by nebulized inhalation, eye drops, or oral ingestion should be at an amount sufficient to produce blood levels of the granulation inhibitor or inhibitor candidate similar to those reached using systemic injection. The amount of granulation inhibitor or inhibitor candidate that must be delivered by nebulized inhalation, eye drops, or oral ingestion to attain these levels is dependent upon the nature of the inhibitor used and can be determined by routine experimentation. For systemic injection of the antibody granulation inhibitor M200, therapeutic levels of the inhibitor were detected in the blood one week after delivery of a 15 mg/kg dose (FIG. 13B). FIGS. 13E and 13F show that repeated dosing with M200, 15 and 50 respectively at weekly intervals, is sufficient to maintain the plasma concentration of M200 at therapeutic levels. This finding is confirmed in FIGS. 14 and 15, which show M200 saturation of α5β1 integrin receptors of plasma macrophage on all days tested (FIG. 14), blocking binding of the anti α5β1 integrin antibody IIA1 (FIG. 15).

Evaluation of granulation levels is determined by staining fixed tissue sections taken from the treated eyes. Briefly, formalin fixed eyes were cut horizontally so that pupil, optic nerve and macula are in the same plane and embedded in paraffin. Serial sections were made through the entire specimen and slides in defined distances were routinely stained with Heamtoxolin and Eosin. Lesions were identified by light microscopy, measured and a map was generated showing the location of lesions, macula and optic nerve. On slides that show histologically the most servere degree of injury (considered to be the center area of the lesion) the area of granulation tissue was measured using the AxioVision software from Carl Zeiss. Inc.

Results obtained from treating the eyes of monkeys using the embodiment described above and in more detail in example 4 produced a dramatic decrease in the amount of scarring produced at the wound site treated with the granulation inhibitor F200 (also referred to as “EOS200-F”), compared to controls, at both 25 μg and 100μg per eye doses (see FIG. 12A). The invention also provides a qualitative assay for detecting granulation inhibitors, based on the quantitative screening assay detailed above. This method of evaluating a granulation inhibitor involves first creating lesions in an eye tissue and applying one or more doses of the granulation inhibitor to the eye tissue as described above. Using the techniques described above, the level of granulation or scarring at the wound site is monitored periodically or at the end of treatment.

High Throughput Techniques

While the methods noted above can be used to identify any type of granulation inhibitor, they are best suited for screening granulation inhibitor candidates that are suspected as being granulation inhibitors, usually through some relationship to known granulation inhibitors (e.g., by belonging to the same chemical family or sharing some other structural or functional feature with a known granulation inhibitor. Moreover, novel granulation inhibitors may be identified using a process known as computer, or molecular modeling, as discussed below.

Computer Modeling

Computer modeling technology allows visualization of the three-dimensional atomic structure of a selected molecule and the rational design of new compounds that will interact with the molecule. The three-dimensional construct typically depends on data from x-ray crystallographic analyses or NMR imaging of the selected molecule. The molecular dynamics require force field data. The computer graphics systems enable prediction of how a new compound will link to the target molecule and allow experimental manipulation of the structures of the compound and target molecule to perfect binding specificity. Prediction of what the molecule-compound interaction will be when small changes are made in one or both requires molecular mechanics software and computationally intensive computers, usually coupled with user-friendly, menu-driven interfaces between the molecular design program and the user.

An example of the molecular modelling system described generally above consists of the CHARMm and QUANTA programs, Polygen Corporation, Waltham, Mass. CHARMm performs the energy minimization and molecular dynamics functions. QUANTA performs the construction, graphic modelling and analysis of molecular structure. QUANTA allows interactive construction, modification, visualization, and analysis of the behavior of molecules with each other.

A number of articles review computer modeling of drugs interactive with specific proteins, such as Rotivinen, et. al., Acta Pharmaceutica Fennica 97, 159-166 (1988); Ripka, New Scientist 54-57 (Jun. 16, 1988); McKinaly and Rossmann, Annu. Rev. Pharmacol. Toxiciol. 29, 111-122 (1989); Perry and Davies, OSAR: Ouantitative Structure-Activity Relationships in Drug Design pp. 189-193 (Alan R. Liss, Inc. 1989); Lewis and Dean, Proc. R. Soc. Lond. 236, 125-140 and 141-162 (1989); and, with respect to a model receptor for nucleic acid components, Askew, et al., J. Am. Chem. Soc. 111, 1082-1090 (1989). Askew et al. constructed a new molecular shape which permitted both hydrogen bonding and aromatic stacking forces to act simultaneously. Askew et al. used Kemp's triacid (Kemp et al., J. Org. Chem. 46:5140-5143 (1981)) in which a U-shaped (diaxial) relationship exists between any two carboxyl functions. Conversion of the triacid to the imide acid chloride gave an acylating agent that could be attached via amide or ester linkages to practically any available aromatic surface. The resulting structure featured an aromatic plane that could be roughly parallel to that of the atoms in the imide function; hydrogen bonding and stacking forces converged from perpendicular directions to provide a microenvironment complimentary to adenine derivatives.

Computer modelling has found limited use in the design of compounds that will interact with nucleic acids, because the generation of force field data and x-ray crystallographic information has lagged behind computer technology. CHARMm has been used for visualization of the three-dimensional structure of parts of four RNAs, as reported by Mei, et al., Proc. Natl. Acad. Sci. 86:9727 (1989). For methods of modelling interactions with nucleic acids, see U.S. Pat No: 6,446,032, and the references therein.

Other computer programs that screen and graphically depict chemicals are available from companies such as BioDesign, Inc., Pasadena, Calif., Allelix, Inc, Mississauga, Ontario, Canada, and Hypercube, Inc., Cambridge, Ontario. Although these are primarily designed for application to drugs specific to particular proteins, they can be adapted to design of drugs specific to regions of RNA, once that region is identified.

Screening Compound Libraries

Whether identified from existing granulation inhibitors or from molecular modelling techniques, granulation inhibitors generally must be modified further to enhance their therapeutic usefulness. This is typically done by creating large libraries of compounds related to the granulation inhibitor, or compounds synthesized randomly, based around a core structure. In order to efficiently screen large and/or diverse libraries of granulation inhibitor candidates, a high throughput screening method is necessary to at least decrease the number of candidate compounds to be screened using the assays described above. High throughput screening methods involve providing a combinatorial chemical or peptide library containing a large number of potential therapeutic compounds (potential modulator or ligand compounds). Such “combinatorial chemical libraries” or “candidate libraries” are then screened in one or more assays, as described below, to identify those library members (particular chemical species or subclasses) that are able to inhibit granulation and limit scar formation. The compounds thus identified can serve as conventional “lead compounds” or can themselves be used as potential or actual therapeutics.

Candidate compounds of the library can be any small chemical compound, or a biological entity, such as a protein, sugar, nucleic acid or lipid, as described previously. Typically, test compounds will be small chemical molecules and peptides. The assays discussed below are designed to screen large chemical libraries by automating the assay steps and providing compounds from any convenient source to assays, which are typically run in parallel (e.g., in microtiter formats on microtiter plates or similar formats, as depicted in FIG. 15, in robotic assays). It will be appreciated that there are many suppliers of chemical compounds, including Sigma (St. Louis, Mo.), Aldrich (St. Louis, Mo.), Sigma-Aldrich (St. Louis, Mo.), Fluka Chemika-Biochemica Analytika (Buchs Switzerland) and the like.

Accordingly, the present invention provides methods for high throughput screening of granulation inhibitor candidates. The initial steps of these methods allow for the efficient and rapid identification of combinatorial library members that have a high probability of being granulation inhibitors. These initial steps take advantage of the observation that granulation inhibitors are also integrin binding agents. Any method that determines the ability of a member of the library, termed a binding candidate, to specifically bind to α5β1 integrin is suitable for this initial high throughput screening. For example, competitive and non-competitive ELISA-type assays can be utilized.

A competitive ELISA assay would include an α5β1 integrin bound to a solid support. The α5β1 integrin would first be incubated with a binding agent from a combinatorial library. After sufficient time to allow the binding agent to bind the α5β1 integrin, the substrate would be washed followed by challenge with a known α5β1 integrin ligand, such as fibronectin. The number of α5β1 integrin binding sites available will be directly proportional to the ability of fibronectin to bind the immobilized α5β1 integrin. If there are few α5β1 integrin binding sites available, it is because the biding sites are occupied by the binding candidate. Binding candidates that are able to block fibronectin binding to α5β1 integrin would be granulation inhibitor candidates. Bound fibronectin may be determined by labeling the fibronectin, as described in Harlow & Lane, Antibodies, A Laboratory Manual (1988).

An exemplary non-competitive assay would follow the same procedure described for the competitive assay, without the addition of a known α5β1 integrin ligand. Binding of the binding candidate to the immobilized α5β1 integrin may be determined by washing away unbound binding candidate; eluting bound binding candidate from the support, followed by analysis of the eluate; e.g., by mass spectroscopy, protein determination (Bradford or Lowry assay, or Abs. at 280 nm determination.). Alternatively, binding may be identified by monitoring changes in the spectroscopic properties of the organic layer at the support surface. Methods for monitoring spectroscopic properties of surfaces include, but are not limited to, absorbance, reflectance, transmittance, birefringence, refractive index, diffraction, surface plasmon resonance, ellipsometry, resonant mirror techniques, grating coupled waveguide techniques and multipolar resonance spectroscopy, all of which are known to those of skill in the art.

Binding candidates that are found to bind α5β1 integrin with acceptable specificity, e.g., with a Kₐ for α5β1 integrin of at least about 10⁵ mol⁻¹, 10⁶ mol⁻¹ or greater, preferably 10⁷ mol⁻¹ or greater, more preferably 10⁸ mol⁻¹ or greater, and most preferably 10⁹ mol⁻¹ or greater, are granulation inhibitor candidates and are screened further, as described above, to determine their ability to inhibit cellular granulation and limit scar tissue formation.

A number of well-known robotic systems have been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, HewlettPackard, Palo Alto, Calif.), which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

V. Therapeutic Uses

Individuals to be treated using methods of the present invention can be any individual having a wound susceptible to collateral tissue damage and/or excessive scarring as a result of pronounced cellular granulation. Such an individual can be, for example, a vertebrate such as a mammal, including a human, dog, cat, horse, cow, or goat; a bird; or any other animal, particularly a commercially important animal or a domesticated animal.

To this end, the current invention provides methods of reducing or inhibiting granulation in a tissue in an individual, by administering to the individual an α5β1 integrin binding agent that is a granulation inhibitor. By reducing granulation, the methods of the present invention limit the amount of scar tissue formed at a wound site and reduce collateral tissue damage caused by swelling and excessive macrophage behavior.

Methods of the present invention are suitable for use on any tissue susceptible to injury or disease that may result in tissue granulation. Such tissues include, but are not limited to, eye, skin, bone, cartilage, vascular, ligament and tendon. Diseases treatable by the present methods include, but are not limited to, rheumatoid Arthritis, temporal arteritis, polymyalgia rheumatica, giant cell arteritis, Takayasu's arteritis, Kawasaki's disease, Wegener's, granulomatosis, Churg-Strauss alleric granulomatosis and angiitis, idiopathic pulmonary fibrosis, systemic sclerosis/scleroderma, Sjogren's syndrome/disease, sicca syndrome, allergic pulmonary fibrosis, sarcoidosis, uterine fibroids, hemangioma, lymphangioma, keloid scar formation, Goodpasteur disease, Crohns disease, Pagets syndrome, pterygiae, midline granuloma, desmoid, macular degeneration, proliferative vitreoretinopathy, proliferative diabetic retinopathy, allergic pulmonary fibrosis and eosinophilic granulomata.

Some embodiments of the methods described herein are particularly suited for treatment of eye injuries and diseases. It has been observed that retinal pigment epithelium (RPE) cells have the ability to take on macrophage-like characteristics, termed macrophage behavior, in response to eye injuries sufficient to induce granulation. This macrophage behavior of RPE cells leads to cell damage surrounding the wound site resulting in excessive scar tissue being formed as well as tissue damage that can result in blindness. Using the granulation inhibitors identified by the methods of the present invention, RPE cells can be coaxed to take on fibroblast-like behavior. The concomitant reduction in macrophage behavior reduces scarring, granulation and much of the consequential tissue damage. As a result, the healed tissue is much more functional that tissue allowed to heal in the absence of a granulation inhibitor (see FIG. 12).

Accordingly, the present invention also provides methods for controlling RPE cell behavior comprising contacting a wound site in an affected eye with an a5b 1 integrin binding agent, preferably a granulation inhibitor, wherein RPE cells in the affected eye are inhibited from displaying macrophage behavior.

In therapeutic use granulation inhibitors generally will be in the form of a pharmaceutical composition containing the inhibitor and a pharmaceutically acceptable carrier. Pharmaceutically acceptable carriers are well known in the art and include aqueous solutions such as physiologically buffered saline or other buffers or solvents or vehicles such as glycols, glycerol, oils such as olive oil or injectable organic esters. The selection of a pharmaceutically acceptable carrier will depend, in part, on the chemical nature of the inhibitor, for example, whether the inhibitor is an antibody, a peptide or a nonpeptide, small organic molecule.

A pharmaceutically acceptable carrier may include physiologically acceptable compounds that act, for example, to stabilize the granulation inhibitor or increase its absorption, or other excipients as desired. Physiologically acceptable compounds include, for example, carbohydrates, such as glucose, sucrose or dextrans, antioxidants, such as ascorbic acid or glutathione, chelating agents, low molecular weight proteins or other stabilizers or excipients. One skilled in the art would know that the choice of a pharmaceutically acceptable carrier, including a physiologically acceptable compound, depends, for example, on the route of administration of the granulation inhibitor and on its particular physio-chemical characteristics.

The methods of the present invention include application of granulation inhibitors in cocktails including other medicaments, for example, antibiotics, fungicides, and anti-inflammatory agents. Alternatively, the methods may comprise sequential dosing of an afflicted individual with a granulation inhibitor and one or more additional medicaments to optimize a treatment regime. In such optimized regimes, the medicaments, including the granulation inhibitor may be applied in any sequence and in any combination.

Cellular granulation resulting from injury or disease can occur locally, for example, in the retina of an individual suffering from diabetic retinopathy, or more systemically, for example, in an individual suffering from rheumatoid arthritis. Depending on the tissue to be treated and the nature of the disease or injury, one skilled in the art would select a particular route and method of administration of the granulation inhibitor. For example, in an individual suffering from diabetic retinopathy, the inhibitor can be formulated in a pharmaceutical composition convenient for use as eye drops, which can be administered directly to the eye. In comparison, in an individual suffering from osteoarthritis, the inhibitor may be delivered in a pharmaceutical composition that can be administered intravenously, orally or by another method that distributes the agent systemically. Thus, a granulation inhibitor can be administered by various routes, for example, intravenously, orally, or directly into the region to be treated, for example, intrasynovially where the condition involves a joint. The granulation inhibitors of the present invention may also be included in slow release formulations for prolonged treatment following a single dose. In one embodiment, the formulation is prepared in the form of microspheres. The microspheres may be prepared as a homogenous matrix of a granulation inhibitor with a biodegradable controlled release material, with optional additional medicaments as the treatment requires. The microspheres are preferably prepared in sizes suitable for infiltration and/or injection, and injected systemically, or directly at the wound site.

Examples of anatomical locations amenable to direct application of the formulation include the vitreous humor of the eye, and intra articular joints including knee, elbow, hip, sternoclavicular, temporomandibular, carpal, tarsal, wrist, ankle, and any other joint subject to arthritic conditions; examples of bursae where the formulations useful in the invention can be administered include acromial, bicipitoradial, cubitoradial, deltoid, infrapatellar, ishchiadica, and other bursa known to those skilled in the art to be subject to formation of deleterious granulation.

The formulations of the invention are also suitable for administration in all body spaces/cavities, including but not limited to pleura, peritoneum, cranium, mediastinum, pericardium, bursae or bursal, epidural, intrathecal, intraocular, etc.

Some slow release embodiments include polymeric substances that are biodegradable and/or dissolve slowly. Such polymeric substances include polyvinylpyrrolidone, low- and medium-molecular-weight hydroxypropyl cellulose and hydroxypropyl methylcellulose, cross-linked sodium carboxymethylcellulose, carboxymethyl starch, potassium methacrylate-divinylbenzene copolymer, polyvinyl alcohols, starches, starch derivatives, microcrystalline cellulose, ethylcellulose, methylcellulose, and cellulose derivatives, β-cyclodextrin, poly(methyl vinyl ethers/maleic anhydride), glucans, scierozlucans, mannans, xanthans. alzinic acid and derivatives thereof, dextrin derivatives, glyceryl monostearate, semisynthetic glycerides, glyceryl palmitostearate, glyceryl behenate, polyvinylpyrrolidone, gelatine, agnesium stearate, stearic acid, sodium stearate, talc, sodium benzoate, boric acid, and colloidal silica.

Slow release agents of the invention may also include adjuvants such as starch, pregelled starch, calcium phosphate mannitol, lactose, saccharose, glucose, sorbitol, microcrystalline cellulose, gelatin, polyvinylpyrrolidone. methylcellulose, starch solution, ethylcellulose, arabic gum, tragacanth gum, magnesium stearate, stearic acid, colloidal silica, glyceryl monostearate, hydrogenated castor oil, waxes, and mono-, bi-, and trisubstituted glycerides

Slow release agents may also be prepared as generally described in WO 94/06416.

In stromal-type tumors, the a5b1 integrin binding agent should be an antibody, preferably an IgGI antibody. IgGI recruits additional beneficial mechanisms in addition to α5β1 antagonism, e.g., complement fixation, ADCC, and T cell recruitment, which would be valuable in treating these tumors

The amount of granulation inhibitor administered to an individual will depend, in part, on the disease and extent of tissue injury. Methods for determining an effective amount of an agent to administer for a diagnostic or a therapeutic procedure are well known in the art and include phase I, phase II and phase III clinical trials. Generally, an agent antagonist is administered in a dose of about 0.01 to 200 mg/kg body weight when administered systemically, and at a concentration of approximately 1 μM, when administered directly to a wound site. The total amount of granulation inhibitor can be administered to a subject as a single dose, either as a bolus or by infusion over a relatively short period of time, or can be administered using a fractionated treatment protocol, in which the multiple doses are administered over a more prolonged period of time. One skilled in the art would know that the concentration of a particular granulation inhibitor required to provide an effective amount to a region or regions of tissue injury depends on many factors including the age and general health of the subject as well as the route of administration, the number of treatments to be administered, and the nature of the inhibitor, including whether the inhibitor is an antibody, a peptide, or a non-peptide small organic molecule. In view of these factors, the skilled artisan would adjust the particular dose so as to obtain an effective amount for efficaciously inhibiting granulation and scar formation for therapeutic purposes.

A granulation inhibitor identified by the methods of the present invention, or a pharmaceutical composition thereof containing the inhibitor, can be used for treating any pathological condition that is characterized, at least in part, by excessive granulation and scar formation. One skilled in the art would know that the inhibitor can be administered by various routes including, for example, orally, or parenterally, including intravenously, intramuscularly, subcutaneously, intraorbitally, intracapsularly, intrasynovially, intraperitoneally, intracistemally or by passive or facilitated absorption through the skin using, for example, a skin patch or transdermal iontophoresis. Furthermore, the inhibitor can be administered by injection, intubation, via a suppository or topically, the latter of which can be passive, for example, by direct application of an ointment or powder containing the inhibitor, or active, for example, using a nasal spray or inhalant for nebulized inhalation delivery. The pharmaceutical composition also can be incorporated, if desired, into liposomes, microspheres or other polymer matrices (Gregoriadis, Liposome Technology, Vol. 1 (CRC Press, Boca Raton, Fla. 1984), which is incorporated herein by reference). Liposomes, for example, which consist of phospholipids or other lipids, are nontoxic, physiologically acceptable and metabolizable carriers that are relatively simple to make and administer.

All publications and patent applications cited in this specification are herein incorporated by reference as if each individual publication or patent application were specifically and individually indicated to be incorporated by reference.

Although the foregoing invention has been described in some detail by way of illustration and example for clarity and understanding, it will be readily apparent to one of ordinary skill in the art in light of the teachings of this invention that certain changes and modifications may be made thereto without departing from the spirit and scope of the appended claims.

As can be appreciated from the disclosure provided above, the present invention has a wide variety of applications. Accordingly, the following examples are offered for illustration purposes and are not intended to be construed as a limitation on the invention in any way. Those of skill in the art will readily recognize a variety of noncritical parameters that could be changed or modified to yield essentially similar results.

### EXAMPLES

### Example 1

### Construction of M200 Chimera from Murine IIA1 Anti-α5β1 Integrin

This example describes construction of the chimeric antibody M200.

A. Starting DNA Sequences of IIA1 and 200-4 VH and VL Domains

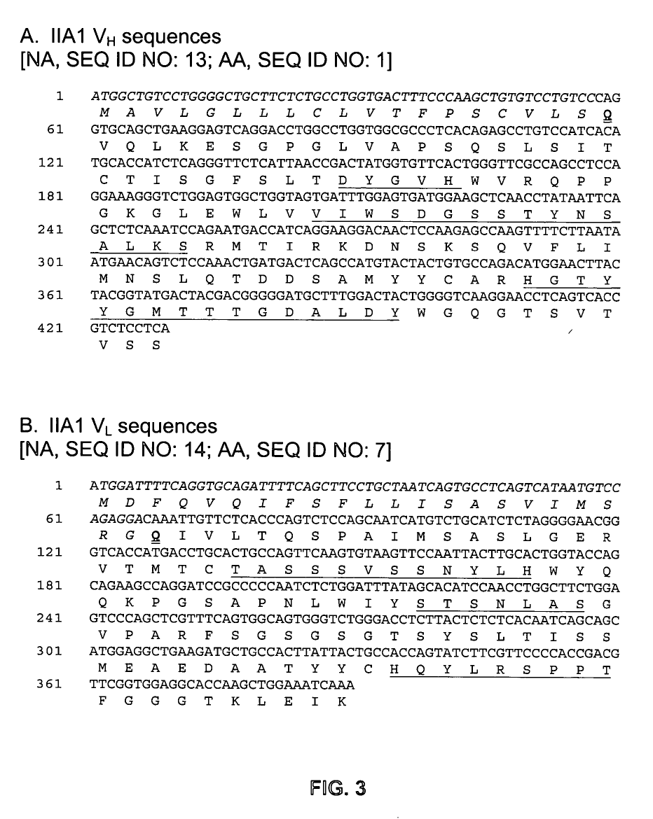

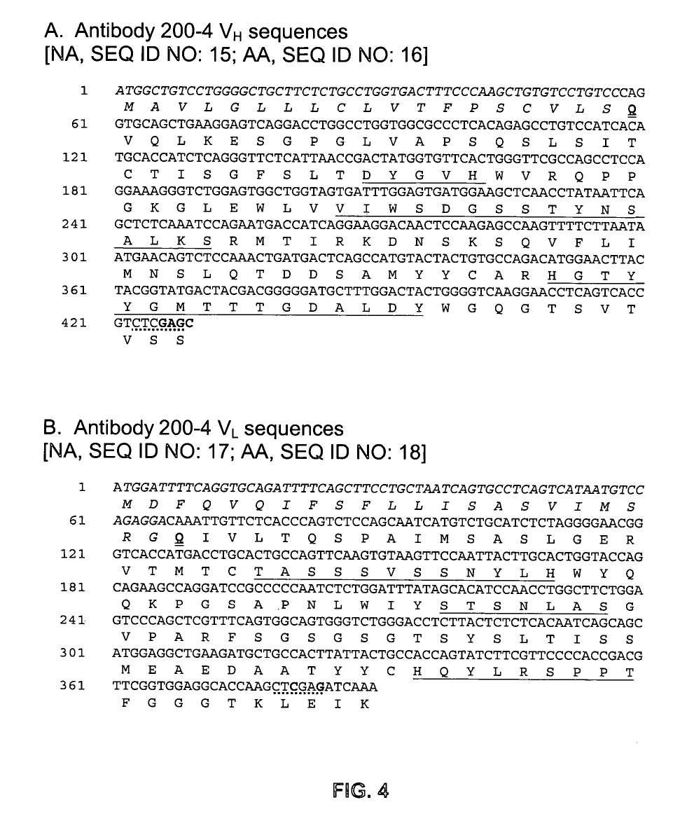

The variable heavy (V_{H}) and light (V_{L}) domains of the mouse anti-human α5β1 integrin antibody, IIA1 (Pharmingen, San Diego Calif.) were cloned from the IIA1 hybridoma cDNA and sequenced as part of the initial construction of the 200-4 antibody. FIG. 3 shows the cDNA sequences of the IIA1 V_{H} (SEQ ID NO: 13) and V_{L} (SEQ ID NO: 14) domains. During the construction of the 200-4 mouse/human chimeric IgG4 antibody from IIA1, silent XhoI restriction sites (CTCGAG) (SEQ ID NO: 29) were introduced into the framework 4 regions of both IIA1 V_{H} and V_{L}. The 200-4 V_{H} (SEQ ID NO: 15) and V_{L} (SEQ ID NO: 17) DNA sequences containing these silent XhoI sites, as found in expression constructs DEF38 IIA1/human G4 chimera and NEF5 IIA1/K chimera, are shown in FIG. 4. These 200-4 V_{H} and V_{L} sequences were used as the starting point for all subsequent recombinant DNA manipulations.

B. Design of M200 VH and VL mini-exons

The 200-4 V_{H} and V_{L} domains in expression plasmids DEF38 IIA1/human G4 chimera and NEF5 IIA1/K chimera are directly fused to their adjacent constant domains through silent XhoI sites, with no intervening introns. In order to make these variable domains compatible with the desired antibody expression vectors based on the genomic DNA, it was necessary to design ‘mini-exons’ which recreate functional donor splice sites at the 3′ ends of the variable coding region. Sequence comparisons revealed that the V_{H} and V_{L} regions of IIA1 utilized the murine JH4 and JK1 segments, respectively; therefore the mini-exons were designed to recreate natural murine JH4 and JK1 donor splice sites following the last amino acid in the V_{H} and V_{L} domains. In addition, the XhoI sites were removed, restoring the framework 4 sequences as found in the original IIA1 hybridoma. The mini-exons were flanked with restriction sites: 5′ and 3′ XbaI sites (TCTAGA) (SEQ ID NO: 30) for the VH mini-exon, and 5′ MluI (ACGCGT) (SEQ ID NO: 31) and 3′ XbaI (TCTAGA) (SEQ ID NO: 30) for the V_{L} mini-exon.

Recombinant antibody variable domains occasionally contain undesired alternative mRNA splice sites, which can then give rise to alternately spliced mRNA species. Such sites could, in theory, exist in the murine variable domain but only become active in the context of a heterogeneous expression cell and/or new acceptor sites from chimeric constant regions. Taking advantage of codon degeneracy to remove potential alternative splice sites while leaving the encoded amino acid sequence unchanged may eliminate such undesired alternative splicing. To detect any potential alternative splice sites in the M200 V_{H} and V_{L} mini-exons, the initial designs were analyzed with a splice site prediction program from the Center for Biological Sequence Analysis from the Technical University of Denmark (http://www.cbs.dtu.dk/services/NetGene2/). For both 200-M mini-exons, the correct donor splice sites were identified; however, potential alternative donor splice sites were detected in CDR3 of the V_{H} mini-exon and CDR1 of the V_{L} mini-exon. To eliminate the possibility of these splice sites being used, single silent base pair changes were made to the mini-exon designs. In the case of the V_{H} design, a silent GGT to GGA codon change at glycine 100 (Kabat numbering) was made; for the V_{L} design, a silent GTA to GTC codon change at valine 29 was made. In both cases these silent changes eliminated the potential secondary splicing donor signal in the V-genes.

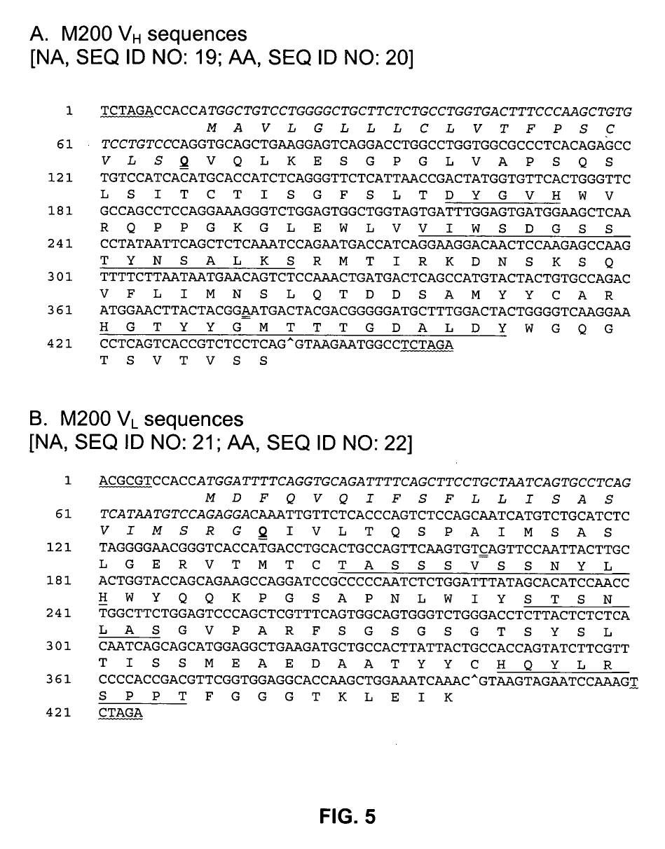

Final designs for the M200 V_{H} and V_{L} mini-exons (SEQ ID NOS: 19, 21), containing the flanking restriction sites, murine donor splice sites, with the 200-4 XhoI sites removed, and with the potential alternative donor splice sites eliminated are shown in FIG. 5.

C. Construction of M200 V_{H} Mini-exon and Plasmid p200-M-H

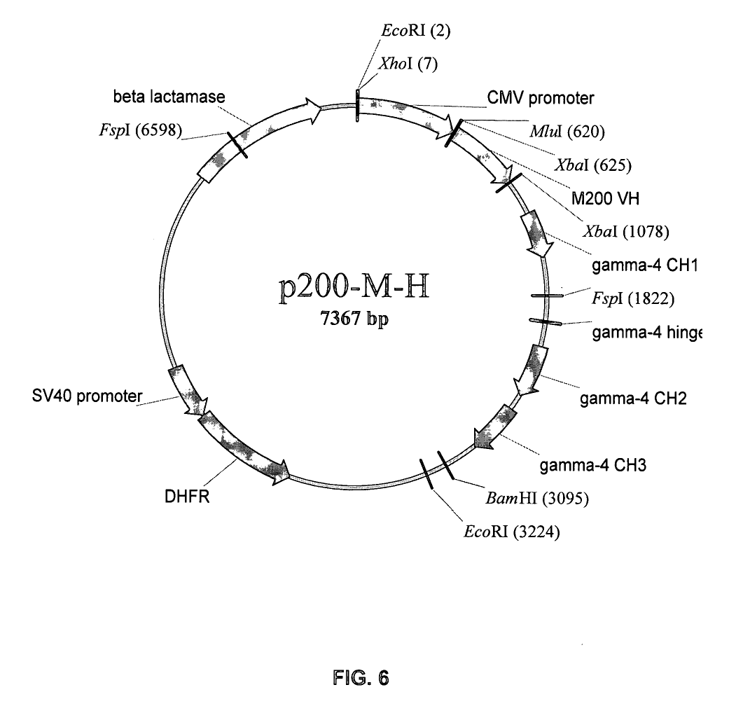

The designed mini-exon for M200 V_{H} as shown in FIG. 5A was constructed by PCR-based mutagenesis using 200-4 expression plasmid DEF38 IIA1/human G4 chimera as the starting point. Briefly, the 200-4 V_{H} region was amplified from DEF38 IIA1/human G4 chimera using primers #110 (5′-TTTTCTAGACCACCATGGCTGTCCTGGGGCTGCTT-3′) (SEQ ID NO: 32), which anneals to the 5′ end of the 200-4 V_{H} signal sequence and appends a Kozak sequence and XbaI site, and primer #104 (5′-TTTTCTAGAGGTTGTGAGGAC TCACCTGAGGAGACGGTGACTGAGGT-3′) (SEQ ID NO: 33) which anneals to the 3′ end of the 200-4 V_{H} and appends an XbaI site. The 469 bp PCR fragment was cloned into pCR4Blunt-TOPO vector (Invitrogen) and confirmed by DNA sequencing to generate plasmid p200M-VH-2.1. This intermediate plasmid was then used in a second PCR mutagenesis reaction to remove the potential aberrant splice site in CDR3 and to add a murine JH4 donor splice site at the 3′ end of the V_{H} coding region. Two complementary primers, #111 (5′-TGGAACTTACTACGGAATGACTA CGACGGGG-3′) (SEQ ID NO: 34) and #112 (5′-CCCCGTCGTAGTCATTCCGTAGTAAGTTCCA-3′) (SEQ ID NO: 35) were designed to direct a GGT to GGA codon change at glycine 100 (Kabat numbering) in CDR3 of the M200 V_{H}. Primers #110 and #112 were used in a PCR reaction to generate a 395 bp fragment from the 5′ end of the M200 VH mini-exon, and a separate PCR reaction with primers #111 and #113 (5′-TTTTCTAGAGGCCATTCTTACCTGAGGAGACGGTGACTGAGGT-3′) (SEQ ID NO: 36) generated a 101 bp fragment from the 3′ end of the M200 V_{H} mini-exon. The two PCR products were gel purified on 1.5% low melting point agarose, combined, and joined in a final PCR reaction using primers #110 and #113. The final 465 bp PCR product was purified, digested with XbaI, and cloned into XbaI-digested and shrimp alkaline phosphatase-treated vector pHuHCg4.D. The final plasmid, p200-M-H (FIG. 6) was subjected to DNA sequencing to ensure the correct sequence for the 200-M V_{H} mini-exon between the XbaI sites and to verify the correct orientation of the XbaI-XbaI insert.

D. Construction of M200 V_{L} Mini-exon and Plasmid p200-M-L

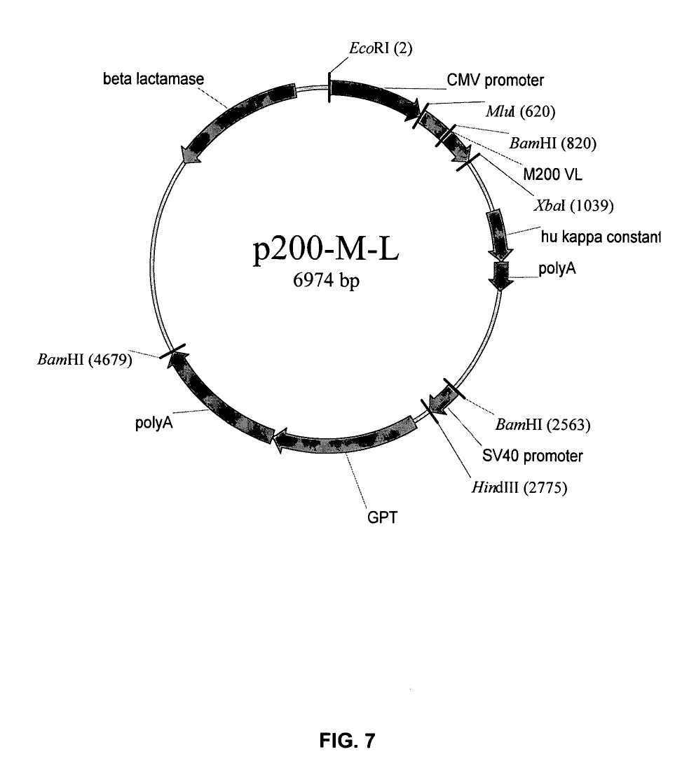

The designed mini-exon for M200 V_{L} as shown in FIG. 5B was constructed by PCR-based mutagenesis using 200-4 expression plasmid NEF5 IIA1/K as the starting point. The V_{L} region was amplified from NEF5-IIA1-K using primers #101 (5′-TTTACGCGTCC ACCATGGATTTTCAGGTGCAGATT-3′) (SEQ ID NO: 37) which anneals to the 5′ end of the signal sequence and appends a Kozak sequence and MluI site, and primer # 102 (5′-TTTTCTAGATTAGGAAAG TGCACTTACGTTTGATTTCCAGCTTGGTGCC-3′) (SEQ ID NO: 38) which anneals to the 3′ end of the 200-4 V_{L} and appends an XbaI site. The 432 bp PCR fragment was cloned into pCR4Blunt-TOPO vector (Invitrogen) and confirmed by DNA sequencing to generate plasmid p200M-V_{L}-3.3. This intermediate plasmid was then used in a second PCR mutagenesis reaction to remove the potential aberrant splice site in CDR1 and to add a murine JK1 donor splice site at the 3′ end of the V_{L} coding region. Two complementary primers, #114 (5′-TGCCAGTTCAAGTGTCAGTTCCAATTACTTG-3′) (SEQ ID NO: 39) and #115 (5′-CAAGTAATTGGAACTGACACTTGA ACTGGCA-3′) (SEQ ID NO: 40) were designed to direct a GTA to GTC codon change at valine 29 (Kabat numbering) in CDR1 of the V_{L} domain. Primers #101 and #115 were used in a PCR reaction to generate a 182 bp fragment from the 5′ end of the V_{L} mini-exon, and a separate PCR reaction with primers #114 and #116 (5′-TTTTCTAGACTTTGGATTCTACTTAC GTTTGATTTCCAGCTTGGTGCC-3′) (SEQ ID NO: 41) generated a 280 bp fragment from the 3′ end of the V_{L} mini-exon. The two PCR products were gel purified on 1.5% low melting point agarose, combined, and joined in a final PCR reaction using primers #101 and #116. The final 431 bp PCR product was purified, digested with MluI and XbaI, and cloned into MluI- and XbaI-digested light chain expression vector pHuCkappa.rgpt.dE. The final plasmid, p200-M-L (FIG. 7) was subjected to DNA sequencing to ensure the correct sequence for the V_{L} mini-exon between the MluI and XbaI sites.

E. Combination of Plasmids p200-M-H and p200-M-L to make Final Expression Plasmid p200-M

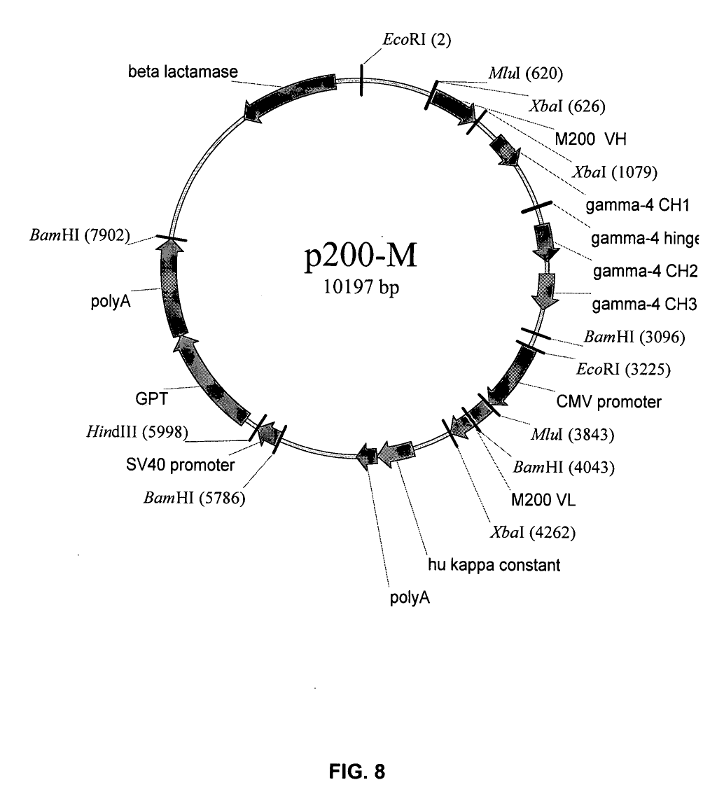

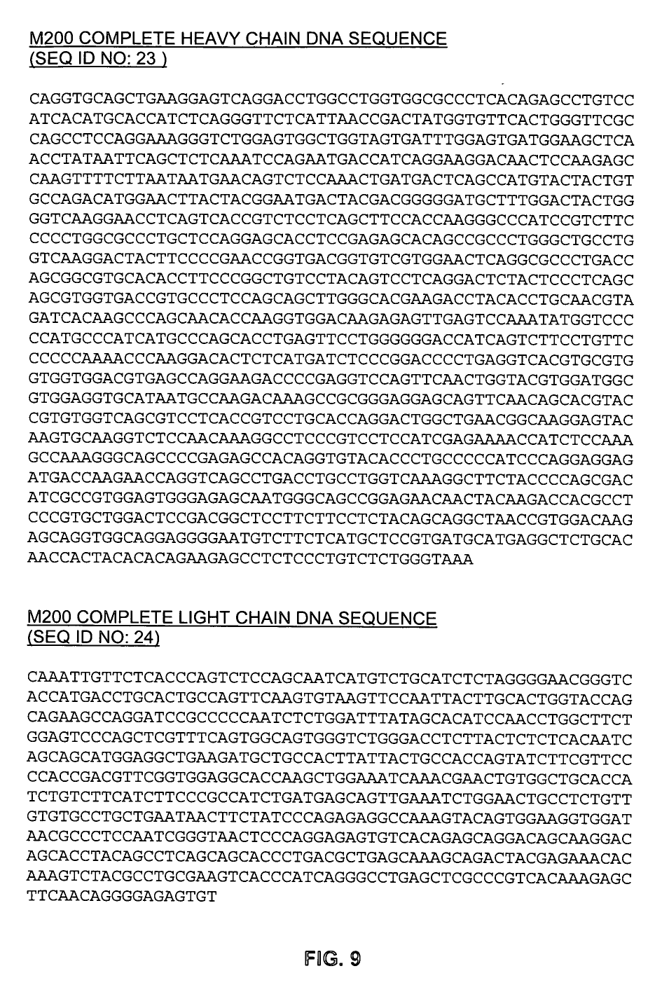

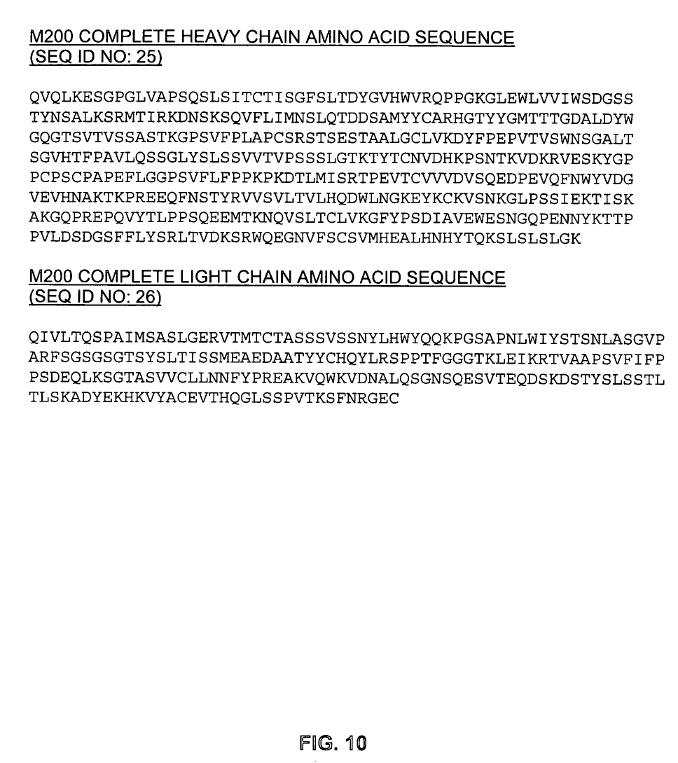

To express M200 from a single plasmid, p200-M-H and p200-M-L were digested with EcoRI, and the EcoRI fragment carrying the entire IgG4 heavy chain gene from p200-M-H was ligated into EcoRI-linearized p200-M-L to generate plasmid p200-M (FIG. 8). A large scale endotoxin-free plasmid preparation of p200-M was prepared from 2.5 liters of E. coli culture using the Endotoxin-Free Plasmid Maxi-prep Kit (Qiagen). The plasmid structure was verified by restriction enzyme mapping with enzymes BamHI, XbaI, and FspI. The entire coding region for M200 V_{H}, V_{L}, Cκ, and Cγ4 were verified by DNA sequencing. The DNA sequences for the complete M200 heavy (SEQ ID NO: 23) and M200 light (SEQ ID NO: 24) chains are shown in FIG. 9. The corresponding amino acid sequences for the complete M200 heavy (SEQ ID NO: 25) and M200 light (SEQ ID NO: 26) chains are shown in FIG. 10.

### EXAMPLE 2

### Generation of Fab Fragment F200 from M200

This example describes making Fab fragment F200.

Fab fragments are generated from M200 IgG starting material by enzymatic digest. The starting IgG is buffer exchanged into 20 mM sodium phosphate, 20 mM N-acetyl cysteine pH 7.0. Soluble papain enzyme is added, and the mixture is rotated at 37° C. for 4 hours. After digestion the mixture is passed over a protein A column to remove Fc fragments and undigested IgG are removed. Sodium tetrathionate is added to 10 mM and incubated for 30 minutes at room temperature. Finally, this preparation is buffer exchanged into 20 mM sodium phosphate, 100 mM sodium chloride, pH 7.4, to yield the F200 solution.

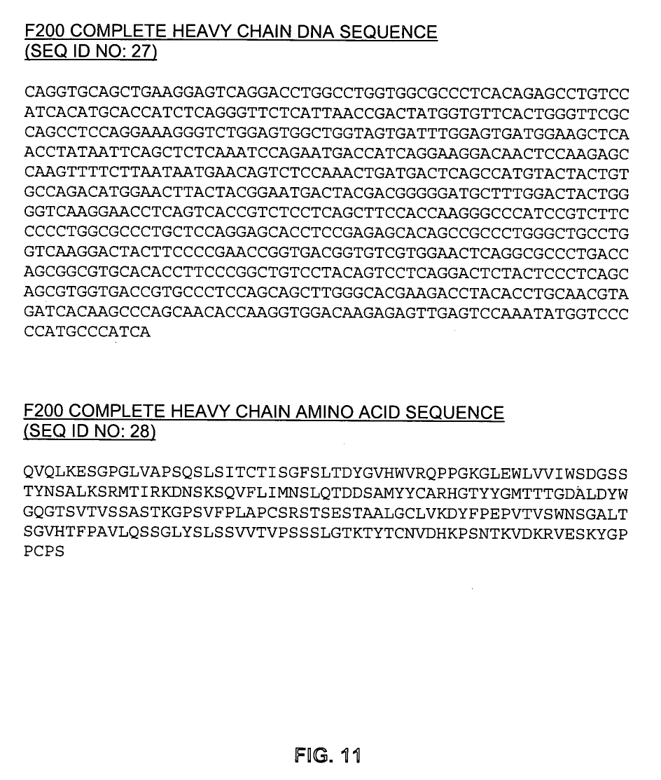

Because it is a Fab fragment, the F200 light chain DNA and amino acid sequences are the same as the M200 light chain. The complete F200 heavy chain DNA (SEQ ID NO: 27) and amino acid (SEQ ID NO: 28) sequences are shown in FIG. 11.

### EXAMPLE 3

### Maintenance of Granulation Inhibitor Serum Levels After Systemic Administration

This example shows that granulation inhibitor serum levels can be maintained through a regular dosing regime.

The dosing of each subject was through systemic delivery by intravenous infusion in the cephalic or saphenous vein. The dose volume for each animal was based on the most recent body weight measurement and was 50, 15 or 5 mg/kg. Intravenous infusion was conducted while the animals were restrained in primate chairs, using syringe infusion pumps. The animals were not sedated for dose administration. The dose schedule was once weekly for 4 weeks beginning on the day of laser injury.

TABLE 1

| | # of | Route of | | | | |
|---|---|---|---|---|---|---|
| Group | animals | administration | Pretreatment | Treatment | Dose | Dosing |
| 1 | 3 | IV | lasered | Vehicle | NA | 4 doses, weekly |
| 2 | 1 | IV | lasered | M200 | 5 mg/kg | 4 doses, weekly |
| 3 | 1 | IV | lasered | M200 | 15 mg/kg | 4 doses, weekly |
| 4 | 3 | IV | lasered | M200 | 50 mg/kg | 4 doses, weekly |

The degree of saturation of α5β1 sites on CD14⁺ monocytes following intra venous administration of M200 was then measured. Using a 2-color assay in which CD14⁺ monocytes are identified using FITC-conjugated anti-CD14, and bound M200 quantified using a PE-conjugated mouse anti-human IgG₄ antibody, providing a measurement of the occupied α5β1 sites. In parallel, the cells are incubated with the murine antibody, IIA1, conjugated to PE, and IIA1 binding is quantified, providing a measurement of unoccupied (available) α5β1 sites. These two measurements are used to calculate the percent saturation of α5β1 sites by M200

Calculation of the degree of saturation is performed by determining the normalized GMF (GMF_{Norm}) of bound M200 using the average GMF (mean fluorescence intensity) value as follows:

$${GMF}_{Norm} M200 = \frac{GMF of PE - anti - human {IgG}_{4}}{GMF of isotype control}$$

Calculate the normalized GMF (GMF_{Norm}) of bound IIA1 using the average GMF value as follows:

$${GMF}_{Norm} IIA1 = \frac{GMF of PE - IIA1}{GMF of isotype control}$$

Calculate the percent occupancy of α5β1 Sites by M200 as follows:

$$\% Occupancy by M200 = ( \frac{( {GMF}_{Norm} M200 - 1 ) \times 100}{( {GMF}_{Norm} M200 - 1 ) + ( {GMF}_{Norm} IIA1 - 1 )} )$$

Results

As shown in FIG. 3A-C, levels of the granulation inhibitor M200 progressively decrease with time. Therapeutic levels of the granulation inhibitor are still present 168hrs after administration of 5 mg/kg of M200, 240hrs, after a 15 mg/kg injection, and more than 336hrs after a 50 mg/kg dose.

FIG. 3D-F illustrates that weekly doses of 15 mg/kg or 50 mg/kg M200 maintains or exceeds the minimum level of granulation inhibitor necessary to provide a beneficial effect. This result is confirmed in M200 and IIA1 binding studies summarized in FIGS. 2 and 3.

FIG. 14 represents the binding of M200 granulation inhibitor to bloodmonocytes. Each bar graph represents the percent occupancy of M200, as determined by FACS analysis, for each day as represented in the accompanying legend. The chart indicates that 4 days after each dose is administered the monocytes α5β1 integrin binding sites are saturated by M200. For the 5 mg/kg dose, levels of M200 binding to monocytes rapidly diminishes, with the levels at day 21 being negligible. However, M200 saturation of monocytes α5β1 integrin binding sites is maintained for the duration of the experiment for both the 15 mg/kg and 50 mg/kg doses. These results are confirmed by the IIA1 FACS analysis depicted in FIG. 15. IIA1 will only bind to monocytes α5β1 integrin binding sites when the sites are not occupied bu M200. As shown in FIG. 15, at the 5 mg/kg dose, binding of IIA1 to monocytes α5β1 integrin binding sites increases to near saturating levels within about 14 days. This increase in IIA1 occupancy tracks the decrease in M200 binding to monocytes α5β1 integrin binding sites. The same pattern of IIA1 binding vs. M200 binding is also observed for the 15 mg/kg and 50 mg/kg doses.

### EXAMPLE 4

### Reduction in Granulation After Intravitreal Treatment with the Granulation Inhibitor F200

This example shows the effect of treating laser-induced eye injuries with the granulation inhibitor F200. Background literature describing studies of laser-induced eye injury in animal models include: S. Ryan, “The Development of an Experimental Model of Subretinal Neovascularization in Disciform Macular Degeneration,” Transactions of the American Ophthalmological Society 77: 707-745 (1979); S. J. Ryan, “Subretinal Neovascularization: Natural History of an Experimental Model,” Archives of Ophthalmology 100: 1804-1809 (1982); M. J. Tolentino et al., “Angiography of Fluoresceinated Anti-Vascular Endothelial Growth Factor Antibody and Dextrans in Experimental Choroidal Neovascularization,” Archives of Ophthalmology 118: 78-84 (2000).

As described below, intravitreal injection of a cellular granulation inhibitor of the present invention significantly reduces tissue granulation at the site of macular lesions induced in monkeys.

Summary

A total of4 monkeys were assigned to treatment groups as shown in the Table 2 below. EOS 200F is a Fab fragment derived from a murine anti-α5β1 integrin IgG and a human IgG, administered in a carrier buffer solution. Macular granulation was induced on Day 1 by laser treatment to the maculae of both eyes of each animal. All animals were dosed as indicated in the table once weekly for 4 weeks. The first day of dosing was designated Day 1. The animals were evaluated for changes in clinical signs, body weight, and other parameters, using standard techniques. All animals were euthanized on Day 32.

TABLE 2

| | Treatment | Treatment | Dose per | |
|---|---|---|---|---|
| Group | (Left Eye) | (Right Eye) | eye | Dosing |
| 1 | Buffer | Buffer | NA | 4 doses weekly |
| 2 | Buffer | Buffer | NA | 4 doses weekly |
| 8 | F200 | F200 | 25 μg | 4 doses weekly |
| 9 | F200 | F200 | 100 μg | 4 doses weekly |

Induction of Cellular Granulation

The animals were fasted overnight prior to laser treatment and dosing. The animals were sedated with ketamine HCl (intramuscular, to effect) followed by a combination of intravenous ketamine and diazepam (to effect) for the laser treatment and dosing procedure.

Macular granulation was induced by laser treatment to the maculae of both eyes. Lesions were placed in the macula in a standard grid pattern with a laser [OcuLight GL (532 nm) Laser Photo-coagulator with a IRIS Medical® Portable Slit Lamp Adaptor]. Laser spots in the right eye mirror placement in the left eye. The approximate laser parameters were as follows: spot size: 50-100 μm; laser power: 300-700 milliwatts; exposure time: 0.1 seconds. Parameters for each animal were recorded on the day of laser treatment. Photographs were taken using a TRC-50EX Retina Camera and/or SL-4ED Slit Lamp, with digital CCD camera.

Dosing

An intravitreal injection of immunoglobulin or buffer control article was performed in each eye. Injection on Day 1 occurs immediately following laser treatment. Prior to dose administration, a mydriatic (1% tropicamide) was instilled in each eye. Eyes were rinsed with a dilute antiseptic solution (5% Betadine solution or equivalent), the antiseptic was rinsed off with 0.9% sterile saline solution (or equivalent) and two drops of a topical anesthetic (proparacaine or equivalent) was instilled in the eye. A lid speculum was inserted to keep the lids open during the procedure and the globe was retracted. The needle of the dose syringe was passed through the sclera and pars plana approximately 4 mm posterior to the limbus. The needle was directed posterior to the lens into the mid-vitreous. Test article was slowly injected into the vitreous. Forceps were used to grasp the conjunctiva surrounding the syringe prior to needle withdrawal. The conjunctiva was held with the forceps during and briefly following needle withdrawal. The lid speculum was then removed. Immediately following dosing, the eyes were examined with an indirect ophthalmoscope to identify any visible post-dosing problems.

A topical antibiotic (Tobrex® or equivalent) can be dispensed onto each eye to prevent infection immediately following dosing and one day after dosing. The animals were returned to their cages when sufficiently recovered from the anesthetic. Dosing was done on a weekly as noted above. The gram amount does levels indicated were for each eye. Concentration ranges for the granulation inhibitors used were as follows: Each intact antibody is used at a concentration of about 1 to about 500 μg/ml, preferably about 10 to about 300 μg/ml, advantageously about 25 to about 200 μg/ml, most preferably 7.5-150 ug/ml of eye. Preferable Fab concentrations are the same as those recited for whole antibodies, most preferably 2.5-50 ug/ml of eye.

Monitoring Inhibition of Granulation

Indirect ophthalmoscopy was used to examine the posterior chamber, and biomicroscopy was used to exam the anterior segment of the eye. The eyes were scored using standard procedures (Robert B. Hackett and T. O. McDonald. 1996, Dermatotoxicology. 5th Edition. Ed. By F. B. Marzulli and H. I. Maibach. Hemisphere Publishing Corp., Washington, D.C.).

The eyes may be photographed (TRC-50EX Retina Camera and/or SL-4ED Slit Lamp, with digital CCD camera). The animals may be lightly sedated with ketamine HCl prior to this procedure, and a few drops of a mydriatic solution (typically 1% tropicamide) was instilled into each eye to facilitate the examination.

Animals were euthanized and the eyes removed and dissected. Formalin fixed eyes were cut horizontally so that pupil, optic nerve and macula are in the same plane and embedded in paraffin. Serial sections were made through the entire specimen and slides in defined distances were routinely stained with Heamtoxolin and Eosin. Lesions were identified by light microscopy, measured and a map was generated showing the location of lesions, macula and optic nerve. On slides that show histologically the most servere degree of injury (considered to be the center area of the lesion) the area of granulation tissue was measured using the AxioVision software from Carl Zeiss. Inc.

Analysis of these groups clearly detected tissue granulation at the lesion sites. As depicted in FIG. 12, granulation in the eyes of treated animals (groups 8 and 9) is significantly reduced in comparison to the level of granulation found in the untreated control animals (groups 1 and 2).

1. A method of controlling macrophage behavior comprising contacting a wound site in an affected eye with an α5β1 integrin binding agent, whereby RPE cells in the affected eye are inhibited from displaying macrophage behavior.

2. The method of claim 1, wherein the binding agent is an anti-α5β1 integrin antibody.

3. The method of claim 2, wherein the antibody comprises a variable heavy chain region having an amino acid sequence homologous to an amino acid sequence selected from the group consisting of SEQ ID NOS.: 1-6, and a variable light chain region having an amino acid sequence homologous to an amino acid sequence selected from the group consisting of SEQ ID NOS.:7-12.

4. The method of claim 1, wherein macrophage behavior comprises phagocytic activity.

5. The method of claim 1, wherein macrophage behavior comprises secreting cytokines, chemokines or mediators of inflammatory responses.

6. The method of claim 1, wherein the wound site is not created by an infection.

7. The method of claim 1, wherein the contacting step comprises a technique selected from the group consisting of direct application, intravitreal injection, systemic injection, nebulized inhalation, eye drop, and oral ingestion

8. A method of reducing deleterious granulation in an injured or diseased tissue comprising applying a α5β1 integrin binding agent to the injured or diseased tissue, whereby the granulation is reduced.

9. The method of claim 8, wherein the α5β1 integrin binding agent is anti-α5β1 antibody comprises a variable heavy chain region having an amino acid sequence homologous to an amino acid sequence selected from the group consisting of SEQ ID NOS.: 1-6, and a variable light chain region having an amino acid sequence homologous to an amino acid sequence selected from the group consisting of SEQ ID NOS.:7-12.

10. The method of claim 8, wherein the tissue is selected from the group consisting of eye, skin, bone, cartilage, vascular, ligament or tendon.

11. The method of claim 8, wherein the diseased tissue is part of an eye, joint or associated with a bursae.

12. The method of claim 8, wherein the injured or diseased tissue is produced by a condition selected from the group consisting of keloid formation, burns and scleroderma.

13. The method of claim 8, wherein the injured or diseased tissue is associated with a disease causing tissue inflammation.

14. The method of claim 13, wherein the disease is selected from the group consisting of rheumatoid arthritis, Wegener's Granulomatosis, Churg-Strauss-allergic granulomatosis, eosinophilic granulomata, midline granuloma, desmoid, sarcoidosis, macular degeneration, proliferative vitreoretinopathy, proliferative diabetic retinopathy, uterine fibroids, arteritis temporalis and Takayasu's arteritis.

15. The method of claim 13, wherein the disease is selected from the group consisting of Crohn's disease, idiopathic pulmonary fibrosis, and allergic pulmonary fibrosis.

16. The method of claim 8, wherein the applying step comprises contacting the binding agent to the tissue by a technique comprising direct application, intravitreal injection, systemic injection, nebulized inhalation, eye drop, or oral ingestion.

17. A method for identifying inhibitors of macrophage behavior in RPE cells comprising:
(a) creating lesions in an eye tissue sufficient to produce granulation;
(b) applying one or more doses of an α5β1 integrin binding agent to the eye tissue; and, (c) monitoring granulation in or around the lesions of the dosed eye tissue;
wherein an increase in fibroblast-like cell behavior indicates an inhibitor of macrophage behavior in RPE cells.

18. The method of claim 17, wherein the eye tissue is a part of the eye of a living primate.

19. The method of claim 17, wherein the monitoring step comprises examining stained tissue sections.

20. The method of claim 17, wherein the eye tissue is selected from the group consisting of retinal, macular and corneal.

21. The method of claim 17, wherein the applying the binding agent comprises a technique selected from the group consisting of direct application, intravitreal injection, systemic injection, nebulized inhalation, eye drop, and oral ingestion.
